SEQUENCE LISTING (31 sequences)

SEQ ID NO: 1 - DNA, 335 bp, Homo sapiens
agtggacactgtgggcatgcgtgtccgtgcagcacatcgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 2 - DNA, 302 bp, Homo sapiens
acatcgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 3 - DNA, 301 bp, Homo sapiens
catcgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 4 - DNA, 298 bp, Homo sapiens
cgccgtgcaggagcctgggggaaggcctttccctcagtgagggttgcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 5 - DNA, 236 bp, Homo sapiens
aacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 6 - DNA, 220 bp, Homo sapiens
gtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 7 - DNA, 321 bp, Homo sapiens
ggagccctcctagtgagtatgaagtgatatctcactgaggttttggtttgcaaaagcaaatgactgatgactaacgatgcaggacatctttccatgtgcatgttggtcatttatatatcttccttggagaaatctctattcagatccttagctcatttttaattgggttatttctcttcttcttgttgagttgtaagagttctttacatattctggatcacagtctcttatcagatatatgatttaaaaatattttctcctagtctgtgagttttttcatttcctagtggtgtccattaaagcacaaaagttttacatgtt

SEQ ID NO: 8 - DNA, 164 bp, Homo sapiens
gaactgcacaatgaccagcaggaggggagaagagagtaggaaaaaggagggaaggacagacatcaagtgccagatgttgtctgaactaatcgagcacttctcaccaaacttcatgtataaataaaatacatatttttaaaacaaaccaataaatggcttacatg

SEQ ID NO: 9 - DNA, 114 bp, Homo sapiens
ggcactgtgctctcggacatcatccagaagtacttcttctccccgacgctcttccgagtcatccgcctggcccgaataggccgcatcctcagactgatccgaggggccaagggg

SEQ ID NO: 10 - DNA, 410 bp, Homo sapiens
gaacagagccgcggagccgagacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 11 - DNA, 250 bp, Homo sapiens
gcttccccacaggcaacgtgaggagagcctgtgcccagaagcaggatgagaagatggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccaga

SEQ ID NO: 12 - DNA, 2066 bp, Homo sapiens
ggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgacgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcact

SEQ ID NO: 13 - DNA, 3497 bp, Homo sapiens
ggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgacgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatg

SEQ ID NO: 14 - DNA, 23 bp, Homo sapiens
catcttccggttcagtgccacca

SEQ ID NO: 15 - DNA, 24 bp, Homo sapiens
gctgccctgcgccactactacttc

SEQ ID NO: 16 - DNA, 24 bp, Homo sapiens
catcccacggcccctgaacaagta

SEQ ID NO: 17 - DNA, 25 bp, Homo sapiens
ctgcgccactactacttcaccaaca

SEQ ID NO: 18 - DNA, 25 bp, Homo sapiens
ggaagagcgtcggggagaagaagta

SEQ ID NO: 19 - DNA, 23 bp, Homo sapiens
atgcacatggaaagatgtcctgc

SEQ ID NO: 20 - DNA, 25 bp, Homo sapiens
tcttggcacttgatgtctgtccttc

SEQ ID NO: 21 - DNA, 22 bp, Homo sapiens
tcatgagggcaaagagcagcgt

SEQ ID NO: 22 - DNA, 24 bp, Homo sapiens
gctgccctgcgccactattacttc

SEQ ID NO: 23 - DNA, 30 bp, Homo sapiens
aagaactcgtcaagaaggcgatagaaggcg

SEQ ID NO: 24 - DNA, 30 bp, Homo sapiens
aggatctcctgtcatctcaccttgctcctg

SEQ ID NO: 25 - DNA, 22 bp, Homo sapiens
aagcaagctacgtgcctggctg

SEQ ID NO: 26 - DNA, 24 bp, Homo sapiens
cagagcccatgtatagttgatttc

SEQ ID NO: 27 - DNA, 18 bp, Homo sapiens
gctgttggcaaaggtctg

SEQ ID NO: 28 - protein, 412 aa, Homo sapiens
GTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 29 - protein, 8 aa, Homo sapiens
GALLVSMK

SEQ ID NO: 30 - protein, 12 aa, Homo sapiens
ELHNDQQEGRRE

SEQ ID NO: 31 - protein, 128 aa, Homo sapiens
NKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKG

(19) United States
(12) Patent Application Publication
(10) Pub. No.: US 2007/0212723 A1
(43) Pub. Date: Sep. 13, 2007

(54) Human sodium channel isoforms

(76) Inventors: Samuel C. Dudley, Atlanta, GA (US); Lijuan Shang, Suwanee, GA (US)

Correspondence Address: BLANK ROME LLP, 600 NEW HAMPSHIRE AVENUE, N.W., WASHINGTON, DC, 20037, US

(21) Appl. No.: 11/707,882

(22) Filed: Feb. 20, 2007

Related U.S. Application Data
(60) Provisional application No. 60/847,084, filed on Sep. 26, 2006.
(60) Provisional application No. 60/774,226, filed on Feb. 17, 2006.

(51) Int. Cl. C12Q 1/68; C07H 21/04

(52) U.S. Cl. 435/6; 435/320.1; 435/325; 536/23.2

(57) ABSTRACT

The present invention relates to novel isoforms in or near the 5′ untranslated region (upstream of the start codon) and the 3′ untranslated region (down stream of the start codon) which correlates with an increased risk of heart disease. These isoforms are various spliced variants of the wild-type sodium channel mRNA. Preferably, the isoforms that correlate with heart disease are E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), and E28D (SEQ ID NO. 9).

This application claims the priority of U.S. Provisional Patent Applications No. 60/847,084, filed Sep. 26, 2006; and 60/774,226, filed Feb. 17, 2006; which are incorporated herein by reference.

This invention was made with United States government support awarded by the National Institute of Health grants HL-64828 and HL73753, a Department of Veterans Affairs Merit grant (SCD), and a National Institute of Health NRSA F32 (AEP) research fellowship. The United States government has certain rights in this invention.

### FIELD OF THE INVENTION

The present invention relates to novel isoforms in and/or near the 5′ untranslated region (upstream of the start codon) and the 3′ untranslated region (downstream of the stop codon) which correlates with an increased risk of heart disease.

### BACKGROUND OF THE INVENTION

Heart disease is the number one cause of death in the United States, surpassing even cancer. The National Center for Chronic Disease Prevention and Health Promotion estimates that approximately 950,000 Americans die of cardiovascular disease every year, accounting for more than 40 percent of all deaths. One form of cardiovascular disease, arrhythmia, is associated with very high levels of morbidity and mortality. Sudden arrhythmic death claims more than 300,000 lives each year.

Arrhythmia is defined as abnormal beating of the heart. Heart beat, a complex process of contraction and expansion, is controlled by electrical impulses, which are, in turn, regulated by the flow of specific ions (K⁺, Na⁺ and Ca⁺²) across cellular membranes. Integral membrane proteins, or channels, act as gates, controlling the flow of ions in and out of cells. Sodium, calcium and potassium channels play pivotal roles in generating cardiac action potential, which triggers contraction. Ion channel dysfunction resulting from genetic mutation is a primary cause of arrhythmia.

Voltage-gated sodium channels are pore-forming membrane proteins responsible for the initiation and propagation of action potentials in excitable membranes in nerve, skeletal muscle and heart cells. The controlled gating of sodium channels in response to membrane depolarizations is necessary for normal electrical signaling and establishing of intercellular communication. The cardiac voltage-sensitive sodium (Na⁺) channel is composed of α and β subunits (FIG. 8). The gene encoding the α-subunit, SCN5A, has been cloned and found to consist of 28 exons spanning over 80 kb of DNA. The α-subunit (or its isoforms) contains four homologous repeated domains (D1-D4), each with six transmembrane segments (S1-S6) (FIG. 8). The α-subunit protein alone forms a functional channel when expressed in mammalian expression systems. The four repeated domains are hypothesized to assemble as a pseudotetrameric structure with the permeation pathway situated at the center. The protein is responsible for the rapid influx of sodium ions that initiate and propagate action potential in the heart and the large peak sodium influxes responsible for excitability and conduction in myocardium and special conduction tissues.

The human voltage-gated cardiac Sodium channel α-subunit, referred to as Nav1.5, which is encoded by the gene SCN5A, is by far the most abundant Sodium channel protein in the human heart. The SCN5A gene has been cloned and characterized in 1992 by Gellens et al. Gellens et al. (Primary Structure and Functional Expression of the Human Cardiac Tetrodotoxin-Insensitive Voltage-Dependent Sodium-Channel. Proceedings of the National Academy of Sciences of the United States of America 89, 554-558 (1992)). SCN5A consists of 28 exons spanning approximately 80 kb found by Wang et al. (Genomic organization of the human SCN5A gene encoding the cardiac sodium channel. Genomics 34, 9-16 (1996)). They described the sequences of all intron/exon boundaries and a dinucleotide repeat polymorphism in intron 16. George et al. (Assignment of the human heart tetrodotoxin-resistant voltage-gated Sodium channel alpha-subunit gene (SCN5A) to band 3p21. Cytogenet. Cell Genet. 68, 67-70 (1995)) mapped the SCN5A gene to 3p21 by fluorescence in situ hybridization, thus making it an important candidate gene for long QT syndrome-3 in 1995. Nav1.5 is responsible for the rapid influx of sodium ions that initiates and propagates action potentials in heart, large peak inward sodium current that underlies excitability and conduction in working myocardium and special conduction tissue. Interventions that modulate sodium current have potent physiologic effects. Mutations in the human SCN5A gene cause the long QT syndrome (LQT) and idiopathic ventricular fibrillation (IVF). Mutations in SCN5A that generate truncated, misprocessed, or dysfunctional proteins produce the Brugada variant of idiopathic ventricular fibrillation. Schott et al. (Cardiac conduction defects associate with mutations in SCN5A. Nat. Genet. 23, 20-21 (1999)) reported a mutation in the SCN5A gene that segregated with progressive cardiac conduction defect (PCCD) in an autosomal dominant manner in a large French family. In a smaller Dutch family, another SCN5A mutation cosegregated with familial nonprogressive conduction defect (Schott et al., 1999). In 2002, Tan et al. (A calcium sensor in the sodium channel modulates cardiac excitability. Nature 415, 442-447 (2002)) demonstrated that calmodulin binds to the carboxy terminal ‘IQ’ domain of the SCN5A in a calcium-dependent manner. This binding interaction significantly enhances slow inactivation, a channel-gating process linked to life-threatening idiopathic ventricular arrhythmias. In addition, multiple lines of evidence indicate that loss of sodium channel function is also highly arrhythmogenic. For example, chronic therapy with sodium channel blocking drugs in patients convalescing from myocardial infarction increased total mortality and sudden cardiac death (SCD), likely due to arrhythmias. Mutations within the SCN5A gene are responsible for a number of cardiac arrhythmias, including Long QT-3 (LQT3), Brugada Syndrome, and isolated cardiac conduction disease (ICCD).

Based on the importance of the SCN5A gene in inherited and possibly acquired heart disease, it is vital to study how sodium channel expression and function are regulated by the promoter region. During the last decade there has been an increasing interest in factors that control the regulation and expression level of ion channels. The promoter regions of various voltage-gated sodium channel types have been elucidated, but only a little about those of Nav1.5. The number of functional channels depends on a multitude of events, i.e. the initiation and regulation of transcription and translation, the translocation and subsequent trafficking, and finally the entrance of the protein into the degradation pathway. Electrophysiological characterization of sodium channels has been performed extensively, but understanding transcription and translation of the sodium channel will open new areas of investigation and allow for genetic screening for arrhythmia in non-coding region of SCN5A.

U.S. Pat. No. 7,094,600 to Wang et al. discloses a method for screening compounds to treat arrhythmia using a mutant sodium channel protein. The mutant protein has an amino acid sequence in which one or more amino acids among the ten amino acids occurring at the carboxy end of the S6 segments of D1, D2, D3 or D4 domains of the sodium channel differs from the amino acid in wild-type sodium channel by substitution with tryptophan, phenylalanine, tyrosine or cysteine.

U.S. Patent Application Publication No. 2003/0157600 to Makielski et al. discloses cDNA sequence and expressed amino acid sequence of a sodium channel α-subunit, called hH1b. A specific mutation in hH1b has been shown to display a different phenotype in relation to a human heart disease when compared other known human sodium channel α-subunit with corresponding mutations.

U.S. Patent No. Patent Application Publication No. 2004/0126787 to Makielski et al. discloses four groups of SCN5A variants that represent the most common SCN5A variants in humans. A specific mutation in one of the variants has been shown to display a different phenotype in relation to a human heart disease when compared other variants and known human sodium channel α-subunit with corresponding mutations.

Despite the large number of arrhythmia-linked SCN5A mutations identified to date, there is a clear need to identify additional arrhythmia-linked genetic mutations as many individuals afflicted with arrhythmia, or with a family history of the disease, test negative for genetic mutations.

### SUMMARY OF THE INVENTION

An aspect of the present invention relates to useful tools for future genetic testing for SCN5A-linked cardiac diseases, such as arrhythmia. Previous searches for SCN5A mutations has focused primarily on coding regions. In contrast, the subject technology focuses analysis in or near the promoter and 5′ and 3′ untranslated regions (UTRs) of the SCN5A gene. Approximately 100 kb of the SCN5A promoter of the human voltage-gated sodium channel has been sequenced. Alterations within the promoter region alters the expression of the SCN5A protein, leading to an arrhythmic state. Significantly, multiple specific 5′ and 3′ mRNA splice variants have also been identified. These variants encode SCN5A isoforms possessing different, cardiac disease-associated, biological activities. Therefore, these findings provide methods and compositions for genetic testing and treatment for SCN5A-linked cardiac diseases, including arrhythmia.

The present inventors have discovered that truncations or splice variants of the 5′ and 3′ mRNA that control sodium channel expression and may be linked to heart disease. The specific variants include the following isoforms: E1B1, E1B2, E1B3, E1B4, E2B1, E2B2, E28B, E28C, and E28D. Isoforms E28B, E28C and E28D are most strongly linked to heart diseases.

Persuant to this discovery, the present invention also provides several methods for using the inventions that are disclosed. One method relates to the detection of the risk of heart disease by detecting the presence of the various splicing variants. These variants are E1B1, E1B2, E1B3, E1B4, E2B1, E2B2, E28B, E28C, or E28D, and most preferably E28B, E28C, or E28D. These variants are compared with their relative abundance in normal individuals, and deviations from the normal abundance indicates an increased risk of heart disease. Preferably, the mRNA truncation sequences are detected using nucleic acid hybridization methods known in the art. mRNA sequencing may also be used to detect the truncation sequences. Other methods of using the invention are also disclosed.

### BRIEF DESCRIPTION OF THE DRAWINGS

FIG. 1 is a schematic representation of the splice variants identified in the 5′ end of the human SCN5A gene. The map shows the genomic structure of SCN5A with untranslated (open bars) or translated (closed bars) transcribed sequences and nontranscribed sequences (lines). Splicing patterns for each of the three exon 1 isoforms are identified.

FIG. 2 are cDNA sequences for E1A, E1B1, E1B2, E1B3, E1B4, E2A, E2B1, and E2B2. The arrow shows where the sequence of each isoform begins.

FIG. 3 is a schematic representation of the splice variants identified in the 3′ end of the human SCN5A gene. Above, the map shows the genomic structure of SCN5A with untranslated (open bars) or translated (closed bars) transcribed sequences and nontranscribed sequences (lines). Splicing patterns for each of the four exon 28 isoforms are identified.

FIG. 4 are cDNA sequences for E28A (FIGS. 4A and 4B), E28B (FIG. 4C), E28C (FIG. 4D), and E28D (FIG. 4E). FIG. 4A shows the 1239 sequences in the translated region of E28A and FIG. 4B shows sequences in the untranslated region (the bold letters shows the short 3′UTR (3′UTR-S) of E28A with 834 bp, while the whole sequence shows long 3′UTR (3′UTR-L) of E28A with 2295 bp. In FIGS. 4C and 4D, the translated sequences for E28B and E28C are shown in bold, while the remaining sequences are untranslated. E28D are contains only translated sequences.

FIG. 5 is a photograph of a gel showing PCR results for detecting the transcription start site (TSS), exon 1 variants of the human cardiac SCN5A gene. Total RNA from fetal and adult heart was used to determinate the TSS and exon 1 isoforms with SCN5A specific primers. RACE-PCR result shows total three hands (380, 200 and 100 bp) in both fetal heart (FH) and adult heart (AH) contained cardiac specific Na+ channel sequences. The first band, 380 bp, corresponds to exon 1A, which is reported previously. A second band, 200 bp, is new exon 1 isoform, referred as to exon 1B (E1B) with multiple TSS, whereas the third 100 bp band is an exon 1 isoform referred as to exon 2B (E2B) with multiple TSS.

FIG. 6 is a photograph of a gel showing RACE-PCR results for detecting the 3′ UTR isoforms of the human cardiac SCN5A gene. Total RNA from human fetal and adult heart was used to determinate the 3′UTR with SCN5A gene specific primers GSP3′. The first two lines are first PCR of RACE product and the last two bands are 2nd RACE-PCR result showing the fetal heart lane (FH) demonstrates three bands, the larger visible band is corresponds to 834 bp short 3′UTR of human cardiac Na+ channel sequences. A second band (˜1.4 kb) is noted in fetal heart (FH) only, whereas the third band is noted in both fetal and adult heart as 0.25 and 0.3 kb bands. The last band is 0.2 kb, which were detected only in adult heart. The presence of four exon 28 isoforms was confirmed by sequencing.

FIG. 7 is a schematic showing alignment of the nucleotide and amino acid sequences (single letter) of the four SCN5A transcriptional isoforms. The isoform name and nucleotide base pairs numbering starting at the initial AUG codon are indicated at the left. The sequences start from exon 27 (shaded) and continue to the poly-A tail. Introns are shown as dashed lines. Splicing of exons B, C, and D result in frame shifts and premature stop codons. Methionine at amino acid 1652 is bolded to indicate the site of introduction of a stop codon in the gene-targeted mouse.

FIG. 8 is a drawing showing the putative secondary structure of the cardiac Na⁺ channel.

FIG. 9 are graphs showing developmental regulation of the human SCN5A exon 28 isoforms. The relative abundances of the four isoforms in fetal (FH, open bars) and adult heart (AH, closed bars) are shown in FIG. 9A. FIG. 9B shows the abundance of each isoform as a percentage of the total SCN5A mRNA. In each case, the full length transcript (E28A) was most prevalent. Exon 28D (E28D) was the second most abundant. Exon 28B (E28B) was least abundant in fetal heart, and exon 28C (E28C) was least abundant in adult heart. E28B and E28D were increased in adult heart when compared to fetal heart. *p<0.05 when comparing FH to AH.

FIG. 10 are graphs showing that C-terminal isoform mRNA abundances vary between control and diseased hearts. Real-time PCR results show that the isoform abundances with respect to the total mRNA abundance in each sample. The changes were determined for the four exon 28 variants in control (black bars) and HF (open bars) patients for total ventricle (FIG. 10A), left ventricle (FIG. 10B), and right ventricle (FIG. 10C). Comparing left and right ventricle in FIG. 10B and FIG. 10C, respectively, shows that the isoform abundance changes are more prominent in the left ventricle. β-Actin was used as a reference in all cases. All mRNA abundances were normalized to one of the normal human ventricular exon 28D mRNA abundances. *p<0.05 when comparing controls to HF hearts.

FIG. 11 are photographs of gels showing tissue-specific expression of human SCN5A exon 28 isoforms. RT-PCR results shows that isoform E28A and D were expressed in skeletal muscle (SKM), as well as in human heart. All four isoforms were found in lymphoblasts. The β-actin was used as internal reference.

FIG. 12 are graphs showing a model truncation that reduces cardiac Na⁺ channel current. In order to test the physiological role of the splice variations, a model truncation was introduced into a single allele of mouse ES cells by gene targeting. FIG. 12A shows cardiomyocytes with and without the altered allele were studied electrophysiologically. Na⁺ channel current-voltage curves are shown for wild-type (black squares) and the truncation (open squares). FIG. 12B shows peak current between the two cell types is compared. Introduction of a truncation in the region of the mRNA splice variations resulted in a substantial reduction in peak Na⁺ current. FIG. 12C shows action potentials (AP) recorded in the current clamp mode from a spontaneously beating wild-type (gray line) and mutant CMs (black line). FIG. 12D compares beating rate, AP amplitude, and AP upstroke velocity between the two types of CMs. The truncation mutant shows a reduced beating rate (beats per second), AP amplitude (mV/10), and maximum AP upstroke velocity (mV/ms), consistent with reduced Na⁺ current. *, p<0.05.

FIG. 13 are graphs showing multi-electrode array (MEA) analysis of differentiated CMs with and without the introduction of the model truncation. FIG. 13A illustrates representative field potentials (FPs) from syncytial wild-type and truncation containing CMs recorded from a single MEA electrode. FIG. 13B compares field potential minima (FPmin) recorded at day 19. The FPmin was significantly decreased by introduction of the truncation. FIG. 13C shows that the time to FPₘᵢₙ (i.e. the field potential rise time, FPᵣᵢₛₑ) was slowed in the truncation CMs as compared to wild-type.

Conduction velocity was decreased substantially in the truncation cardiomyocytes (FIG. 13D). All changes are consistent with a reduced Na⁺ current. *p<0.05 between wild-type and the truncation.

FIG. 14 are graphs showing cell viability testing. FIG. 14A: H9c2 cardiomyocyte viability at 48 h as a function of angiotensin II concentration. FIG. 14B: H9c2 cardiomyocyte viability at 48 h as a function of H₂O₂. *, P<0.05 when compared with 10 μmol/L H₂O₂.

FIG. 15 are graphs showing downregulation of cardiac Na⁺ channel mRNA by AngII and H₂O₂. H9c2 or acutely isolated neonatal cardiomyocytes cultured in serum free media (SFM) or exposed to AngII (100 nM or 2 nM), H₂O₂ (20 μM or 40 nM), or AngII (100 nM or 2 nM) and PEG-catalase (250 U/mL) for 48 h. AngII and H₂O₂ resulted in reduced Na⁺ channel mRNA abundance in both H9c2 (FIG. 15A) and neonatal cardiomyocytes (FIG. 15B). In both cases, the AngII effect could be prevented by PEG-catalase (AngII+CAT). *, P<0.05.

FIG. 16 are graphs showing that cardiac Na⁺ channel current is downregulated by H₂O₂. FIG. 16A shows representative Na⁺ currents recorded from H9c2 CMs with voltage steps to −10 mV before and after 48 h of H₂O₂ exposure. FIG. 16B shows that H₂O₂ exposure (20 μM for 48 h) resulted in a 46% (±2.9%, p=0.01) reduction in peak Na⁺ current. *, P<0.05.

FIG. 17 shows that mutation of the NF-κB binding site in the Na⁺ channel promoter eliminates the effects of AngII and H₂O₂. FIG. 17A shows the relationship of the promoter-reporter fragment used to the mouse scn5a promoter (GenBank AY769981). The top line shows the structural organization of this region of the scn5a promoter (3.0 kb). Note the presence of untranslated exon 1C and part of exon 2, which contains the translation start site. Nucleotide numbering starts with +1 corresponding to the protein translation start site. The construct, APS3, containing the NF-κB site (♦) showed reduced activity after 48 h of exposure to 100 nM AngII or 20 μM H₂O₂. Mutating the NF-κB binding site prevented the AngII or H₂O₂ effects (FIG. 17B). Data are presented as mean±S.E.M and are based on 4 separate experiments in both groups.

FIG. 18 are photographs of gels showing that AngII and H₂O₂ induce NF-κB binding to the scn5a promoter. FIG. 18A shows an electrophoretic mobility shift assays with nuclear extracts from H9c2 cardiomyocytes showed that under basal conditions (SFM), there was little binding of NF-κB to the scn5a promoter. H₂O₂ or AngII exposure resulted in the NF-κB binding to the promoter. TNF-α activated H9c2 cell nuclear extract (5 μg) was used as positive control. CAPE (caffeic acid phenethyl ester), a NF-κB inhibitor, prevented binding in response to H₂O₂. AngII-induced NF-κB binding was inhibited by unlabeled probe but not mutant unlabeled probe. FIG. 18B shows that AngII and H₂O₂ promote binding of the p50 subunit of NF-κB to the cardiac Na+channel promoter. Chromosomal immunoprecipitation assay using primers specific for scn5a promoter shows that the p65 subunit of NF-κB appears to be constitutively bound to the channel promoter but that the p50 subunit binds in response to AngII or H₂O₂. CAPE, and inhibitor of NF-κB, could prevent biding of both subunits to the channel promoter. Lanes 1 and 2 are positive and negative controls, respectively. The input DNA was diluted with 1:10.

FIG. 19 shows that overexpression of the p50 NF-κB subunit results in Na⁺ channel transcriptional downregulation. Panel A shows that the presence of the p50 or p65 NF-κB subunit RNA in H9c2 cells stably transfected with vectors encoding p50, p65, or both. Con represents control cells. Panel B: Quantitative real-time RT-PCR result shows the relative scn5a mRNA abundance was decreased in cell lines expressing the p50 subunit in comparison with control (Con). * p<0.05 vs. control.

FIG. 20 is a graph showing a comparison of the abundances of SCN5A splice variants as a function of age. Comparing the relative mRNA abundances of the splice variants after dividing subjects into three age groups of 40-49 (40's), 50-59 (50's) and 60-69 (60's) shows that splice variant abundances did not appear to be a function of age.

FIG. 21 are diagrams showing the targeting strategy to create a mouse SCN5A truncation model. FIG. 21A: Targeting vector pBSK.SCN5A^{1652stop} mapped to the native SCN5A exon 28 region (WT SCN5A allele). FIG. 21B: Map showing incorporation of the targeting vector into the WT allele. FIG. 21C: Map of the truncation mutation introduced into WT SCN5A allele after Cre-mediated excision of the neomycin resistance cassette to create SCN5A^{1652stop}. Restriction digests, PCR primers, and hybridization probes A (3.1-kb PvuII fragment) and B (3.71-kb PvuII fragment) for genotyping are indicated.

FIG. 22 are photographs of gels showing the genotyping for homologous recombination of the SCN5A^{1652stop}. FIG. 22A. PCR analysis using upper and lower PCR amplicons (see methods and figure S2) demonstrates proper recombination in heterozygous (±) and not in wild-type (WT) mice (+/+). FIGS. 22B and 22C: Southern blot analysis with external probes A and B showing proper incorporation of the truncation vector in a single allele of targeted ES cells (±). FIG. 22D: PCR result of a properly targeted ES cell clone before (neo+) and after (neo−) successful excision of the neomycin resistance cassette. The targeting vector was used as a control. FIG. 22E: BspHI restriction digests to demonstrate incorporation of the targeting vector. Introduction of the coding mutation resulted in elimination of a BspHI restriction site. Therefore, the properly targeting allele displayed an additional 545 bp fragment representing the targeted allele (heterozygous, ±) as compared to the 395 bp and 150 bp fragments resulting from the native sequences (wild-type, WT) when performing a BspHI digest of a PCR amplicon spanning this region.

### DETAILED DESCRIPTION OF THE PREFERRED EMBODIMENTS

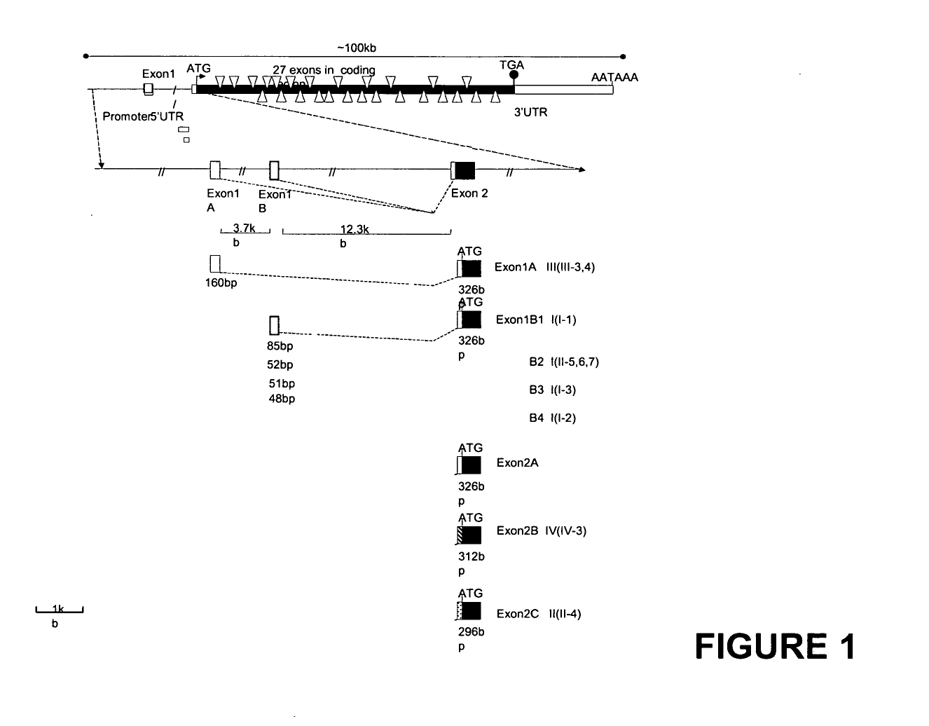

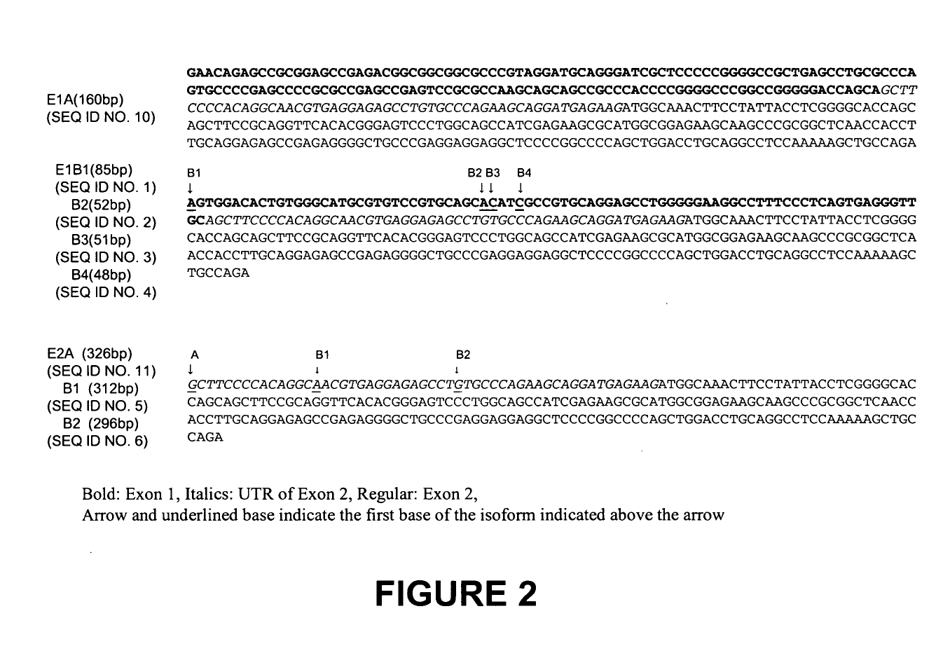

The present invention relates variants (isoforms) in or near the 5′ and 3′ untranslated region (UTR) of the mRNA sequence of SCN5A to heart diseases, such as arrhythmia and heart failure. These variants are preferably E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), or E28D (SEQ ID NO. 9), and most preferably E28B, E28C, or E28D. E1B1, E1B2, E1B3, E1B4, E2B1, and E2B2 are from the 5′ region whose locations in the SCN5A gene and mRNA are depicted in FIG. 1. The nucleic acid sequences for E1B1, E1B2, E1B3, E1B4, E2B1, and E2B2 are shown in FIG. 2. From FIG. 1, E1A (SEQ ID NO. 10) is the wild-type isoform in and/or near the 5′UTR of exon 1, while E1B1,E1B2, E1B3, E1B4 are its various spliced variants. Similarly, E2A (SEQ ID NO. 11) is the wild-type isoform in and/or near the 5′UTR of exon 2, while E2B1, and E2B2 are its various variants.

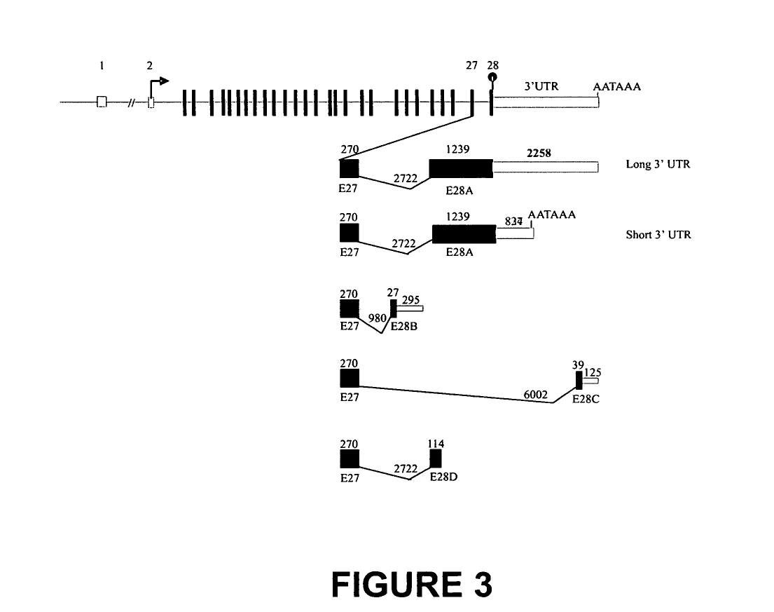

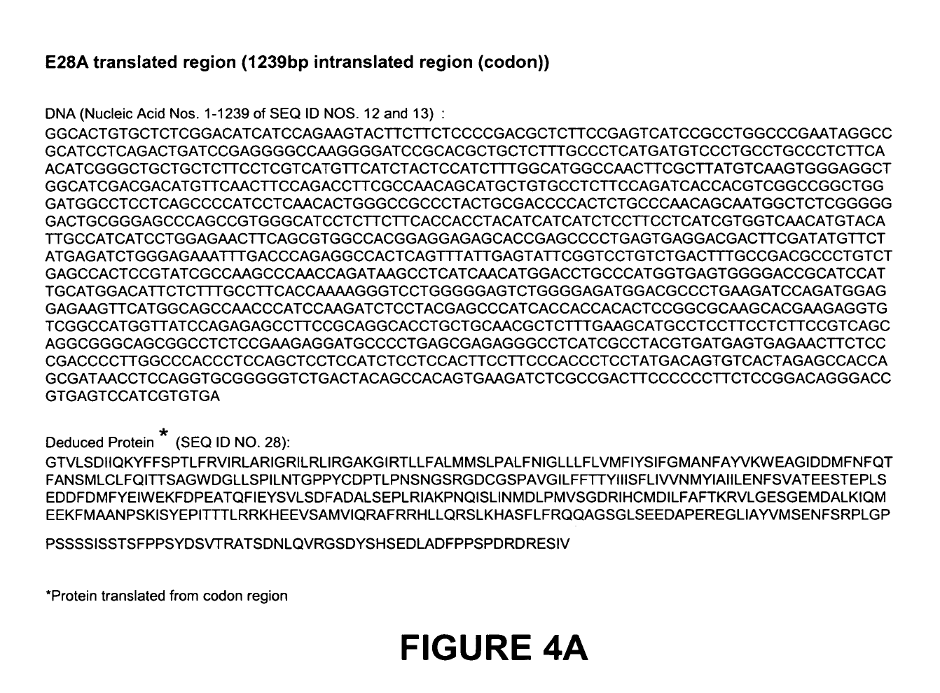

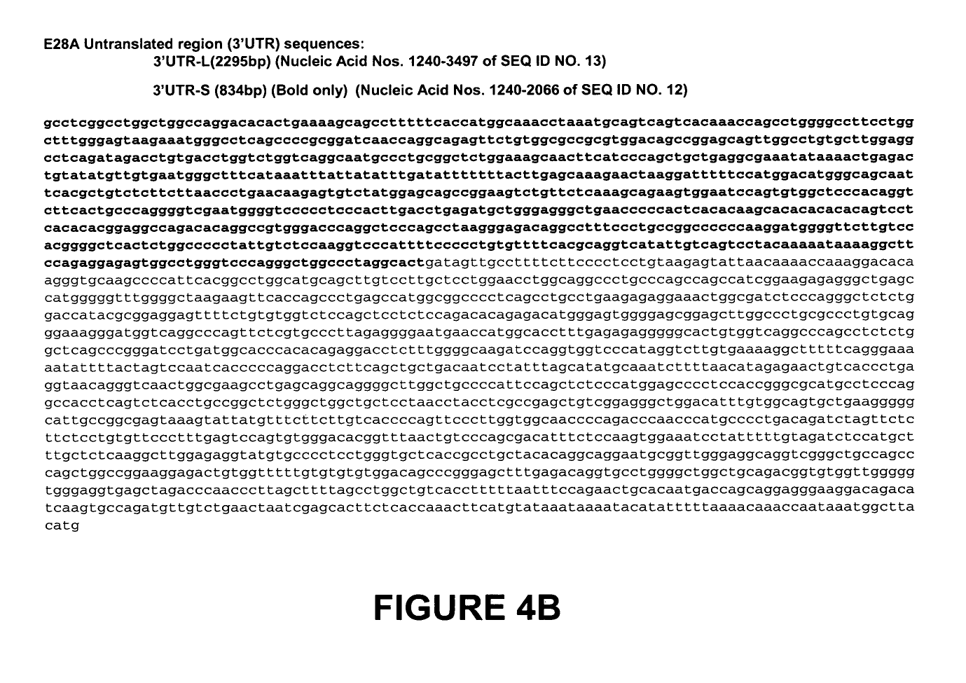

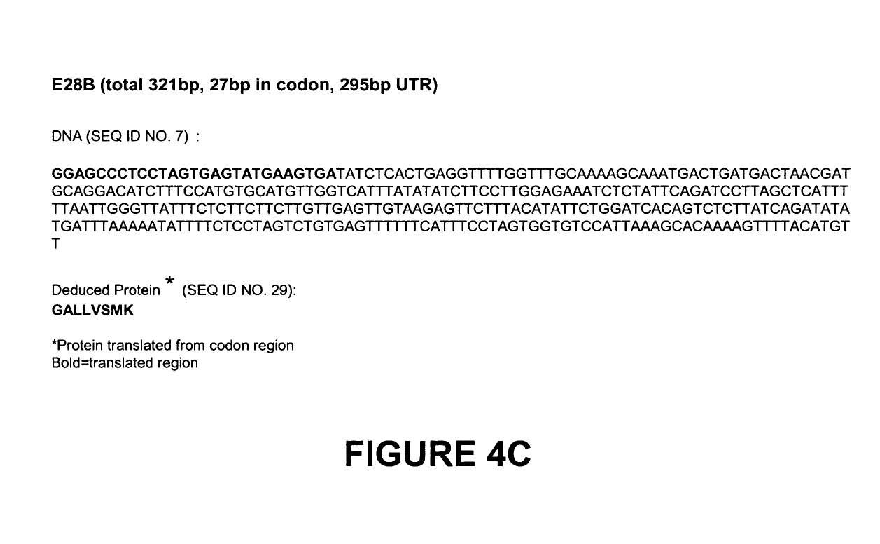

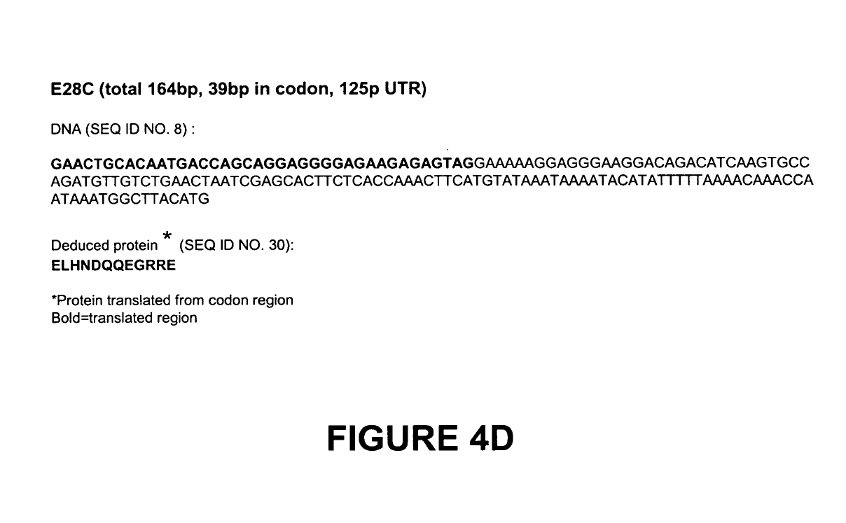

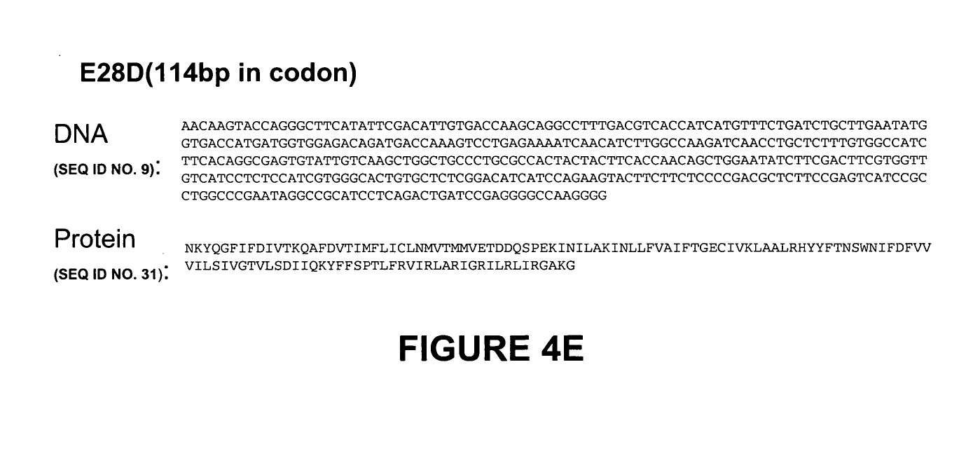

E28B, E28C, or E28D are from or near the 3′ untranslated region whose locations in the SCN5A mRNA are depicted in FIG. 3. The nucleic acid sequences for E28B, E28C, and E28D are shown in FIGS. 4C-4E. From FIG. 3, E28A is the wild-type isoform of the 3′ region of exon 28, while E28B, E28C, or E28D are its various truncated isoforms encoding shortened, dysfunctional channels. There are two isoforms of the E28A: E28A-short (E28A-S) (SEQ ID NO. 12) and E28A-long (E28A-L) (SEQ ID NO. 13). Both isoforms of E28A contains 1239 bp in the translated region (FIG. 4A). The difference between E28-L and E28-S resides in the UTR (FIG. 4B) where E28A-L contains 2295 bp of the 3′UTR, while E28A-S contains 834 bp of the 3′UTR. As shown in FIG. 4B, E28A-S contains only the first 834 bp of the 3′UTR (shown in bold). Both E28B and D28C contains untranslated and translated (codon) regions while E28D contains only translated region of exon 28 (FIGS. 3 and 4E).

Although the isoforms are in or near the UTR, the particular amino acid sequences of coding region are also part of the invention.

In one embodiment, the present invention relates to an isolated polynucleotide having the nucleic acid sequence of the SCN5A variants that are related to heart diseases. The truncations can be of any nucleic acid sequences, especially mRNA, that are related to heart disease. Preferably, theses sequences include at least one of E1B1, E1B2, E1B3, E1B4, E2B1, E2B2, E28B, E28C, and E28D, most preferably at least one of E28B, E28C, and E28D. Preferably, the sequences are identical to those of E1B1, E1B2, E1B3, E1B4, E2B1, E2B2, E28B, E28C, and E28D; however, in certain cases, slight differences from those sequences are also encompassed by the present invention as long as it does not affect the function of the isoform. The difference is preferably limited to 10 or fewer, more preferably 5 or fewer, and most preferably I or fewer amino acid positions. However, it is understood that substitutions can be introduced into non-critical nucleic acid positions and this will not materially affect the function even when more than 20 nucleic acids, preferably 10, more preferably 1, are substituted. A variant with such substitutions is within the scope of the present invention. Additionally, although the truncated variants are discovered in the mRNA of SCN5A, cDNA created therefrom are also within the scope of the present invention.

In another embodiment, the present invention relates to an isolated nucleic acid containing a polynucleotide or its complement, wherein the coding polynucleotide has an uninterrupted sequence that encodes a polypeptide of the invention as set forth above. A nucleic acid containing a polynucleotide that is at least 80% identical to the polynucleotide or its complement over the entire length, or partial length thereof, of the polynucleotide can be used as a probe for detecting the coding polynucleotide and is thus within the scope of the present invention.

In a related aspect, any nucleic acid sequence of the present invention described above can be provided in a vector in a manner known to those skilled in the art. The vector can be a cloning vector or an expression vector. In an expression vector, the polynucleotide is under the transcriptional control of one or more non-native expression control sequences which can include a promoter not natively found adjacent to the polynucleotide such that the encoded polypeptide can be produced when the vector is provided in a compatible host cell or in a cell-free transcription and translation system. Such cell-based and cell-free systems are well known to the skilled artisan. Cells comprising a vector containing a nucleic acid of the invention are themselves within the scope of the present invention. Also within the scope of the present invention is a host cell having the nucleic acid of the present invention integrated into its genome at a non-native site.

In another embodiment, the present invention also includes an isolated nucleic acid probe that contains a fragment of at least 12, 15, 20 or 25 contiguous nucleotides of an SCN5A variant disclosed herein, or its complement. Such a nucleic acid molecule can be used to detect the presence of the SCN5A variant in a cell. The detection reaction can be run under stringent hybridization conditions known in the art. Moderately stringent conditions, which are known in the art, can also be used.

The probe is preferably attached to a label or reporter molecule, may be used to isolate other polynucleotide sequences, having sequence similarity by standard methods. Techniques for preparing and labeling probes are known in the art and disclosed in Sambrook et al.( Molecular Cloning: A Laboratory Manual, Ed.2; Cold Spring Harbor, N.Y.: Cold Spring Harbor Laboratory, 1989) or Ausubel et al. (Current Protocols in Molecular Biology, Wiley & Sons, New York, N.Y, 1995). The labels may be incorporated by any of a number of means well known to those of skill in the art (see U.S. Pat. No. 6,333,155). Commonly employed labels include, but are not limited to, biotin, fluorescent molecules, radioactive molecules, chromogenic substrates, chemiluminescent labels, enzymes, and the like. The methods for biotinylating nucleic acids are well known in the art, as are methods for introducing fluorescent molecules and radioactive molecules into oligonucleotides and nucleotides.

The detection of the SCN5A variant disclosed herein generally comprises (i) amplifying a mRNA from the tissue of an individual, and (ii) hybridizing the amplified fragments with probes specific for the SCN5A isoforms, and (iii) determining the relative amounts of the isoforms based on the degree of hybridization. Nucleic acid samples used in the methods and assays of the present invention may be prepared by any available method or process.

Most preferably polymerase chain reaction (PCR) is used to amplify the nucleic acid (see, e.g., U.S. Pat. Nos. 4,683,203; and 4,683,195, which are incoporated herein by reference). In general, the PCR procedure describes a method of gene amplification which is comprised of (i) sequence-specific hybridization of primers to specific nucleic acid sequences, (ii) subsequent amplification involving multiple rounds of annealing, elongation, and denaturation using a polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified. The primers are prepared using any suitable method, such as conventional phosphotriester or phosphodiester methods or automated embodiments thereof (Beaucage, Tet. Lett. 22:1859-1862, 1981).

Nucleic acid hybridization simply involves contacting a probe and target nucleic acid (from a nucleic acid sample) under conditions where the probe and its complementary target can form stable hybrid duplexes through complementary base pairing (see U.S. Pat. No. 6,333,155, which is incorporated herein by reference). Methods of nucleic acid hybridization are well known in the art. In a preferred embodiment, the probes are immobilized on solid supports such as beads, microarrays, or gene chips.

In a related embodiment, the present invention enables the detection of the risk of heart disease by determining the presence of the mRNA variants, preferably E1B1, E1B2, E1B3, E1B4, E2B1, E2B2, E28B, E28C, or E28D, and most preferably E28B, E28C, or E28D. An increased risk of heart diseases exist when the relative abundance of the variants differs from that of a normal individual. Preferably the presence of the variants are measured in the cells of the heart muscle, skeletal muscle, or the lymphoblasts.

In another embodiment, the present invention provides a method of screening for agents that are capable of modulating heart diseases. A compound may be screened for its ability to affect the binding of NF-κB to the promoter of region of SCN5A. Test compounds that are able to promote the binding of NF-κB to the promoter region is a candidate for treating heart diseases. This screening may be accomplished using high throughput screening methods known in the prior art, such as those disclosed in U.S. Pat. No. 7,091,048 to Parce et al., which is incoporated herein by reference. The screening method normally involve contacting a candidate agent with NF-κB and the promoter of SCN5A. If the agent is able to enhance the strength or degree of binding between NF-κB and the promoter, it is may be used to modulate heart diseases.

Alternatively, an agent may be given to an individual with heart disease and activity of the promoter or changes in splice variation is monitored. If the transcription levels return to levels in normal individual, the agent may be effective to treat heart disease.

In another embodiment, the present invention provides a method for monitoring the treatment of an individual with heart disease. The method comprises administering a pharmaceutical composition to an individual and determining the levels of transcription of the variant mRNA levels. If the levels return to levels in normal individual, the agent is effective in treating the heart disease.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

The human heart tissue from fetal and adult was homogenized, and total RNA isolated using the RNeasy Mini kit following the manufacturer's instructions (Qiagen, Valencia, Calif.). RNA ligase-mediated-rapid amplification cDNA ends (RLM-RACE) methods were used to characterize the 5′ and 3′ ends of the human SCN5A mRNA using GeneRacer kit (Invitrogen, Carlsbad, Calif.). Briefly, 1 μg total RNA was treated with calf intestinal phosphatase to remove the 5′ phosphates of the truncated mRNA and non-mRNA forms of total RNA. Tobacco acid pyrophosphatase was used to remove the 5′ cap structure from intact, full-length mRNA, and T4 RNA ligase was use to add the GeneRacer RNA Oligo to the 5′ end of the mRNA. The first-strand cDNA was synthesized by SuperScript II reverse transcriptase using a reverse gene specific primer GSP5′ (5′CATCTTCCGGTTCAGTGCCACCA 3′ (SEQ ID NO: 14)) complementary to exon 3 of human SCN5A gene and the GeneRacer Oligo dT primer at the 5′ and 3′ ends, respectively. The 5′ and 3′ ends PCR reactions were performed with Platinum Pfx DNA polymerase using 10 μM of GSP5′ and the GeneRacer 5′ primer for amplifying the 5′ end fragment and using 10 μM of the forward GSP3′ (5!GCTGCCCTGCGCCACTACTACTTC3′ (SEQ ID NO: 15)) complementary to exon 27 of the human SCN5A and the GeneRacer Oligo dT primer to obtain 3′ end. An additional PCR reaction with nested primers was performed. The nested PCR products were cloned into pCRII-TOPO vector (Invitrogen, Carlsbad, Calif.) and sequenced to confirm the RACE-PCR products were from the human SCN5A cDNA.

Primary DNA sequence analysis was performed with Vector NTI 7 software (Informax, Frederick, Md.). The sequences were aligned to human genomic DNA and cDNA sequences in Genebank database to identify transcription start sites (TSSs) and 3′ polyadenylylation sites.

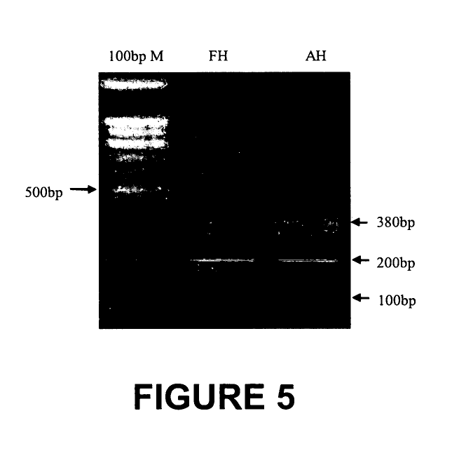

The first human SCN5A cDNA for this channel reported by Sheng et al. (Molecular-Cloning and Functional-Analysis of the Promoter of Rat Skeletal-Muscle Voltage-Sensitive Sodium-Channel Subtype-2 (Rskm2)—Evidence for Muscle-Specific Nuclear-Protein Binding to the Core Promoter. Dna and Cell Biology 13, 9-23 (1994); see also Zhang et al. Dual tandem promoter elements containing CCAC-Like motifs from the tetrodotoxin-resistant voltage-sensitive Sodium channel (rSkM2) gene can independently drive muscle-specific transcription in L6 cells. Gene Expression 8, 85-103 (1999)) (accession # M77235) was 8.5˜9.0 kb, with a 5′ end extending to 150 bp upstream of the ATG codon and 2293 bp 3′ UTR, which contains neither polyadenylylation signal sequence nor a poly A region. The mouse scn5a gene has complex 5′ and 3′ UTR and 5′ and 3′ UTR splice variants are developmentally regulated (Shang, L. L. & Dudley, S. C., Jr. Tandem promoters and developmentally regulated 5′- and 3′-mRNA untranslated regions of the mouse Scn5a cardiac sodium channel. J. Biol. Chem. 280, 933-940 (2005)). Therefore, we undertook to evaluate whether the previously reported human UTR sequences represented the full extent of the cDNA isoforms. The RACE procedure was employed to the 5′ and 3′ mRNA ends upstream of the start codon and downstream of the stop codon, respectively. PCR amplification of cDNA yielded several distinct bands on gel electrophoresis. Subsequent nested PCR amplification gave three bands (380 bp, 200 bp and 100 bp) in both fetal heart RNA and adult heart RNA (FIG. 5). Sequences of these bands showed that the all three bands were SCN5A gene specific. A comparison between the 5′RACE-PCR products and genomic sequences showed that there were two different exon 1 isoforms from reported data, one was 160 bp and located 16.0 kb upstream of exon 2, which is known. A novel isoform was 85 bp, which was separated by 12.3 kb from exon 2. Another new splicing exon 1 was found in exon 2, which means the there is no exon 1 splice. The RNA directly transcripted from exon 2, but the TSS was short 13 bp in comparison with the normal exon 2. From the RACE procedure, we were unable to obtain the exon 1 isoform reported previously (Shang, L. J. & Dudley, S. C. Splice variants of the cardiac Sodium channel 5 ′ UTR differ with development. Biophysical Journal 86, 424A (2004)), suggesting that a total of three exon 1 splice variants existed. And the multiple TSS was existed in all splicing isoforms. We named these untranslated cDNA fragments, exon 1 A (160 bp), exon 1B (85 bp), and exon 2B (313 bp), respectively. Exon 1A, identified by primer extension and RNase protection, has been reported previously in human and rat with a length ranging from 97-176 bp (Yang et al. Cloning and initial characterization of the human cardiac sodium channel (SCN5A) promoter. Cardiovascular Research 61, 56-65 (2004)). All isoforms of the SCN5A 5′UTR were summarized in FIGS. 1 and 3 and sequences were depicted in FIGS. 2 and 4.

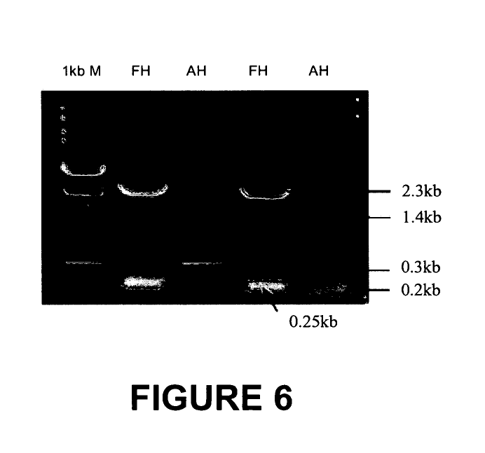

Analysis of the 3′UTR showed evidence of splice variations in fetal and adult heart. By RACE-PCR using GSP3′ and Oligo dT primers, we identified six alternative 3′UTRs (FIG. 6). Comparison of genomic and cDNA sequences showed that the 3′ UTRs had six different poly A splicing variants. One was long, 2295 bp after the stop codon, and was not found out in Genebank, but it is consistent with the published mouse scn5a cDNA (accession # AJ271477). Additionally, there was a second, short variant of 824 bp, which corresponded to the human scn5a cDNA 5′UTR (accession # M77235). These two isoforms have the exon 28 by the same splice referred to as E28A. Three novel exon 28 were detected in the 3′UTR, of those E28B is fetal specific, E28D is adult specific, whereas the E28C expressed in both fetal and human heart. A summary of the findings of the 3′UTR is presented in FIG. 3. The sequences of the all isoforms for the SCN5A 3′UTR were preformatted in FIG. 4.

### EXAMPLE 2

Methods

Detection of Human SCN5A 3′UTR Isoforms by RACE-PCR

Total human RNA from normal fetal and adult whole hearts was purchased from Clontech (Mountain View, Calif.). The RNA ligase-mediated rapid amplification of cDNA ends (RLM-RACE) method was used to characterize the 3′ ends of the human SCN5A mRNA using the GeneRacer kit (Invitrogen, Carlsbad, Calif.). Briefly, 1 μg total RNA was used to synthesize first-strand cDNA by SuperScript II reverse transcriptase using the GeneRacer Oligo dT primer at the 3′ ends. The 3′ ends PCR reactions were performed with Platinum Pfx DNA polymerase using 10 μM of a forward gene-specific primer (GSP) HE26F (5′ CATCCCACGGCCCCTGAACAAGTA 3′ (SEQ ID NO. 16)) complementary to exon 26 of human SCN5A gene and the GeneRacer 3′ primer for amplifying the 3′ end fragment. An additional PCR reaction with a nested GSP primer HE27F (5′ CTGCGCCACTACTACTTCACCAACA 3′ (SEQ ID NO. 17)) corresponding to exon 27 of human SCN5A gene and a nested GeneRacer 3′ primer was performed. The nested PCR products were cloned into pCR4-TOPO vector (Invitrogen, Carlsbad, Calif.) and sequenced. Sequences were compared to that of SCN5A using Vector NTI 7 software (Invitrogen).

Isolation and Culture of Lymphoblasts

Human lymphoblast cell lines were developed from peripheral blood mononuclear cells of volunteers with normal cardiac function referred to the cardiac catheterization laboratory at the Atlanta Veterans Administration Medical Center (AVAMC). Procedures and consent forms were approved by the Emory Institutional Review Board and the AVAMC's Research & Development Committee. To initiate immortalized lymphoblast cell lines, peripheral blood was fractionated by Ficoll-Hypaque centrifugation and mononuclear cells were infected with the B95-8 strain of Epstein-Barr virus (EBV) (39). After EBV-transformation, lymphoblasts were cultured in RPMI-1640 medium supplemented with 10% heat-inactivated fetal bovine serum, 2 mM L-glutamine, 100 U/mL penicillin, and 100 mg/mL streptomycin, at 37° C. in a humid atmosphere with 5% CO₂. Medium was changed twice weekly. Cell counts and viability were assessed by trypan blue staining and fluorescence microscopy. Approximately 5.0×10⁷ lymphoblasts were collected by centrifugation and the RNA isolated using TRIzol reagent (Invitrogen) as described by manufacturer's manual.

Real-Time SYBR Green PCR Quantification of SCN5A Transcript Isoforms

Ventricular tissue from hearts removed at the time of cardiac transplantation at Emory University Hospital under a protocol approved by the Emory Institutional Review Board was homogenized, and total RNA was isolated using the TRIzol reagent (Invitrogen, Carlsbad, Calif.) following the manufacturer's instructions. The total RNA from ventricles and skeletal muscle of the normal adults was bought from Ambion (Austin, Tex.) and Clontech, respectively. To determine the abundance of different cardiac mRNAs carrying variant exon 28 (E28) isoforms, total RNA from left and right ventricles of both normal and patients was used for synthesizing cDNA by reverse transcription using iScript cDNA synthesis Kit (Bio-Rad, Hercules, Calif.) following the manufacturer's instructions. The first strand cDNA was used as template for subsequent PCR. Each PCR reaction contained 12.5 μL of QuantiTect SYBR Green PCR kit master mix (Qiagen, Valencia, Calif.) and 200 nM primer pairs in total 25 μL reaction volume. The reversed primers for exon 28 variants were HSCN5AE28A/R (5′ GGAAGAGCGTCGGGGAGAAGAAGTA 3′ (SEQ ID NO. 18), E28A and 28D), HSCN5AE28B/R (5′ ATGCACATGGAAAGATGTCCTGC 3′ (SEQ ID NO. 19), E28B), HSCN5AE28C/R (5′ TCTTGGCACTTGATGTCTGTCCTTC 3′ (SEQ ID NO. 20), E28C) and HSCN5AE28D/R (5′ TCATGAGGGCAAAGAGCAGCGT 3′ (SEQ ID NO. 21), E28A only), respectively. The forward primer, HE27F, was constant in each case. The reactions gave rise to 124 bp, 170 bp, and 143 bp and 211 bp PCR products, respectively. Amplification with primers HE27F and HSCN5AE28D/R produced the full length isoform, E28A. Amplification with HE27F and HSCN5AE28A/R produced a product comprised of both isoforms E28A and E28D. The amount of E28D was calculated by subtraction of the products of these two reactions. All amplifications were performed in triplicate and consisted of 40 cycles of 30 s at 94° C., 30 s at 65° C., and 1 min at 72° C. in a BioRed thermocycler iCycler (Hercules, Calif.). PCR products were analyzed by electrophoresis on 1.5% agarose gels. β-actin was used as an internal reference when making quantitative comparison.

Generation of Truncated Scn5a Mouse Model

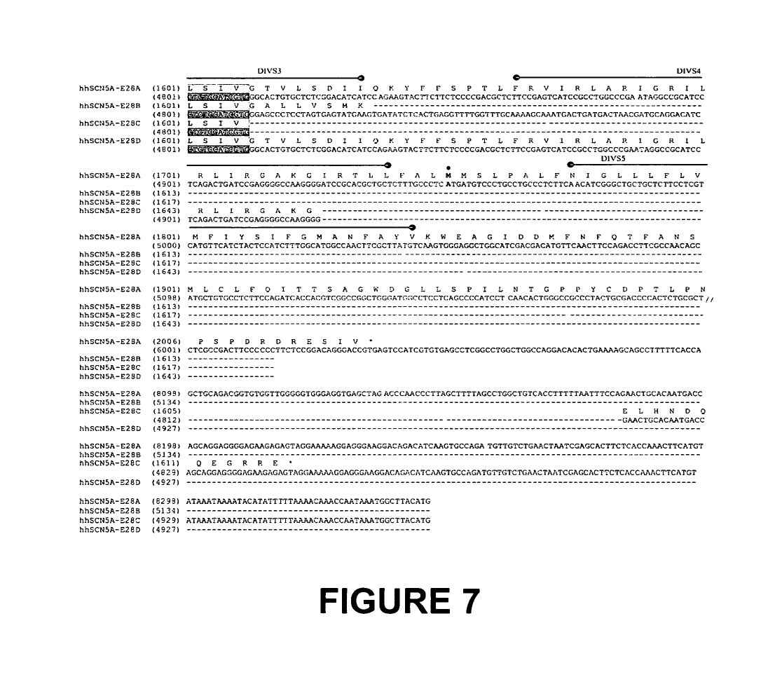

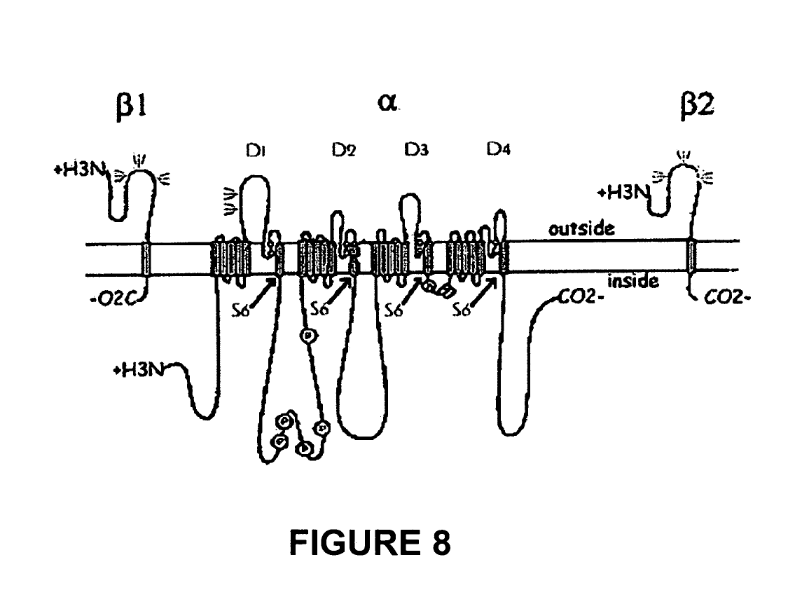

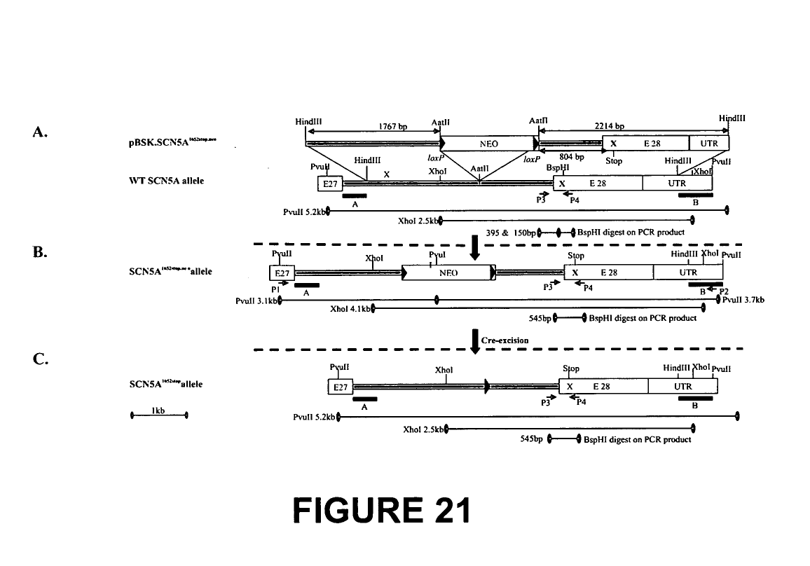

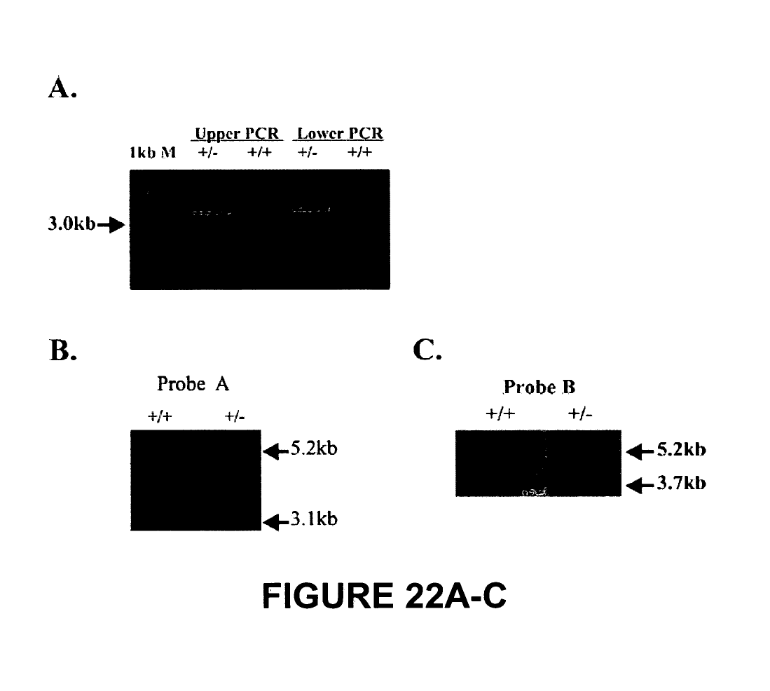

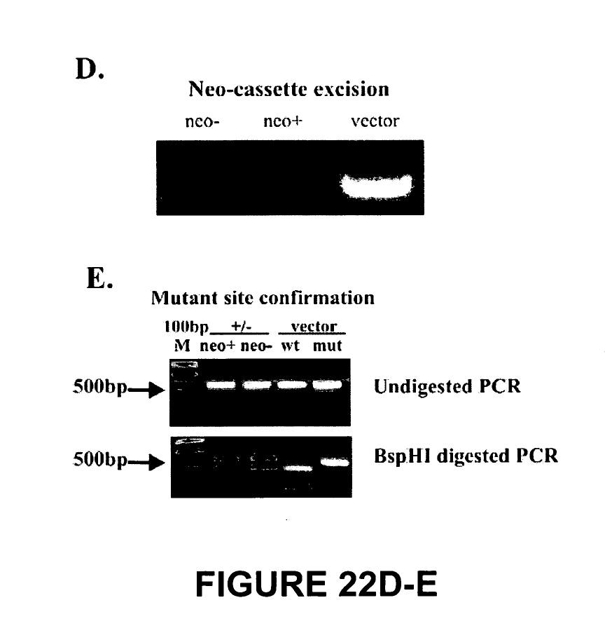

A 4.0-kb fragment of 129Sv/J mouse genomic DNA was cloned from a mouse ES cell genomic library by PCR amplification using primers RHI28F/R (11) corresponding to the known mouse SCN5A exon 28 sequences (40). One PCR positive clone was used to construct the scn5a truncation targeting vector pBSK.SCN5A^{1652stop} by subcloning a 4.0-kb HindIII genomic fragment. The floxed PGK-neomycin cassette was inserted into AatII site (FIG. S2). An adenosine (A) was deleted in this codon. This deletion resulted in a frame shift turning the 1652 codon that formerly encoded for a methionine into a stop codon (TGA; NCBI ACCESSION NP_{—}932173; FIG. 7), predicted to cause premature Sodium channel truncation. The 5′ and 3′ arms of the targeting vector separated by neomycin resistance cassette were 1,767 and 2,214 bp, respectively, leaving a 942 bp homologous fragment on 5′ side of the mutation (FIG. S2). After electroporation with 20 μg of the KpnI-linearized targeting construct into the mouse embryonic stem cells (R1), targeted clones were screened and identified by PCR using primers P1/neoR (P1:5′ GCTGCCCTGCGCCACTATTACTTC 3′ (SEQ ID NO. 22)); neorR: 5′ AAGAACTCGTCAAGAAGGCGATAGAAGGCG 3′ (SEQ ID NO. 23)) neoF/P2 (neorF: 5′ AGGATCTCCTGTCATCTCACCTTGCTCCTG 3′ (SEQ ID NO. 24); P2: 5′ AAGCAAGCTACGTGCCTGGCTG 3′ (SEQ ID NO. 25)) at the 5′ and 3′ ends and neo-cassette (FIGS. 21B and 22A), and using primers P3/P4 (P3: 5′ CAGAGCCCATGTATAGTTGATTTC 3′ (SEQ ID NO. 26); P4: 5′ GCTGTTGGCAAAGGTCTG 3′ (SEQ ID NO. 27)) surrounding the mutation site (FIGS. 21 and 22E). Southern blotting of the 5′ and 3′ ends with the external probes A and B was used to confirm the PCR results (FIGS. 21, 22B, and 22C). The floxed neomycin cassette was excised by expressing transfected Cre recombinase in correctly targeted clones confirmed by PCR using neorF/R (FIG. 21D). Heterozygosity was confirmed by P3-P4 PCR product with or without BspHI digestion (FIG. 21E). Experimental studies were performed on an ES cell line in which one allele of the Sodium channel was successfully targeted.

In vitro Differentiation of ES Cell into Cardiomyocytes

R1 mouse ES cells with or without the mutation were maintained in the undifferentiated state using high glucose Dulbecco modified Eagle medium (DMEM, GibcoBRL, Life Technologies Inc., Rockville, Md.) with supplements and differentiated as described previously (Zhang, Y. M., Shang, L., Hartzell, C., Narlow, M., Cribbs, L., and Dudley, S. C., Jr. 2003. Characterization and regulation of T-type Ca²⁺ channels in embryonic stem cell-derived cardiomyocytes. Am. J. Physiol Heart Circ. Physiol 285:H2770-H2779). For patch clamp experiments, areas of beating CMs were mechanically dissected from 19 day old embryoid bodies, and single CMs were obtained by enzymatic digestion of the dissected areas (Shang et al. 2006. Analysis of arrhythmic potential of embryonic stem cell-derived cardiomyocytes. Methods Mol. Biol. 330:221-231). For the MEA experiments, areas of beating CMs were mechanically dissected from 17 day old embryoid bodies, placed on top of a MEA, and cultured for another 2 days prior to recording.

Recording of Sodium Current from ES-Derived Cardiomyocytes

Patch clamp experiments were performed 1 to 5 days after cell isolation. Cardiomyocytes with uniform contractions and beating rates were used in the study. Patch pipettes were pulled to resistance of 2 to 5 MΩ. Current clamp experiments were conducted in a solution consisting of (in mM) NaCl 140, KCl 5.4, CaCl₂ 1.8, MgCl₂ 1, HEPES 10, and glucose 10 (pH 7.4 by NaOH). The intracellular solution contained (in mM) KCl 120, MgCl₂ 1, MGATP 3, HEPES 10, and EGTA 10 (pH 7.2 by KOH). For voltage clamp experiments, the glass pipettes were filled with a solution of (in mM) CsCl 60, Cesium aspartate 80, EGTA 11, HEPES 10, and Na₂ATP 5 (pH 7.2 with CsOH). The bath solution consisted of (in mM) NaCl 30, N-methyl-D-glucamine (NMDG) Cl 100, CsCl 5, CaCl₂ 2, MgCl₂ 1.2, HEPES 10, and Glucose 5 (pH 7.4 with HCl). Patch clamp data were collected with an Axopatch 200B amplifier and pCLAMP software (Axon Instruments). Data were digitized at 10 kHz and filtered for analysis at 1 kHz. Experiments were performed at 37° C.

Functional Assessment of a Truncation Mutant by Multi-Electrode Array Recording

Extracellular recording from WT and truncation syncytial CMs derived from ES cells was performed using a MEA data acquisition system (Multi Channel System, Reutlingen, Germany). The MEA consists of a matrix of 60 titanium nitride coated gold electrodes (30-μm diameter) in an 8×8 layout grid with an inter-electrode distance of 200 μm. The MEA was inserted in the amplifier system. Simultaneous recordings of bipolar extracellular FPs from all electrodes were performed at a sampling frequency of 10 kHz and at 37° C. One electrode at the border of the array was grounded and used as a reference electrode. As described previously by Jiao et al. (2006. A possible mechanism of halocarbon-induced cardiac sensitization arrhythmias. J. Mol. Cell Cardiol. 41: 698-705), the data were analyzed off-line with a customized toolbox programmed for MATLAB (Mathworks, Natick, Mass.). In order to measure conduction velocity (CV), we calculated the activation time at each point using a threshold-crossing algorithm to form an isochrone map. Three non co-linear points from an area with uniform, parallel isochrones were chosen to calculate CV in two orthogonal directions (e.g. vx and vy). The final CV was calculated as v=((1/vx)²+(1/vy)²)^{-1/2}.

Statistical evaluations

All data are present as means±S.E.M. Statistical analysis of mean values was carried out using unpaired t tests or one-way ANOVAs with post-hoc correction for multiple comparisons. A p value<0.05 was considered statistically significant.

Results

Detection of Three Novel Human SCN5A C-Terminal mRNA Splicing Variants.

Two SCN5A mRNA variants that do not alter the coding sequence have been reported previously from mouse heart that differed in the length of the poly-A tail (Gellens et al. 1992. Primary structure and functional expression of the human cardiac tetrodotoxin-insensitive voltage-dependent sodium channel. Proc. Natl. Acad. Sci. U.S.A 89:554-558; and Shang, L. L., and Dudley, S. C., Jr. 2005. Tandem promoters and developmentally regulated 5′- and 3′-mRNA untranslated regions of the mouse Scn5a cardiac sodium channel. J. Biol. Chem. 280:933-940). Using RACE-PCR, we found analogous sodium channel mRNA isoforms in the human heart (FIG. 6). In addition to these bands, nested RT-PCR revealed shorter bands in both fetal and adult human heart. Sequence analysis of the bands revealed three new mRNA splice variants, as described above and designated as exons E28B (27 bp), E28C (39 bp), and E28D (114 bp) (Genbank accession numbers EF092292, EF092293 and EF092294, respectively). The alternative splicing that produced these novel exons are summarized in FIG. 3. In comparison with the full-length E28A isoform, all three new isoforms were shorter and were predicted to result in prematurely truncated sodium channel proteins missing the segments from domain IV, S3 or S4 to the C-terminus.

The Relative Abundances of the SCN5A Isoforms are Developmentally Regulated.

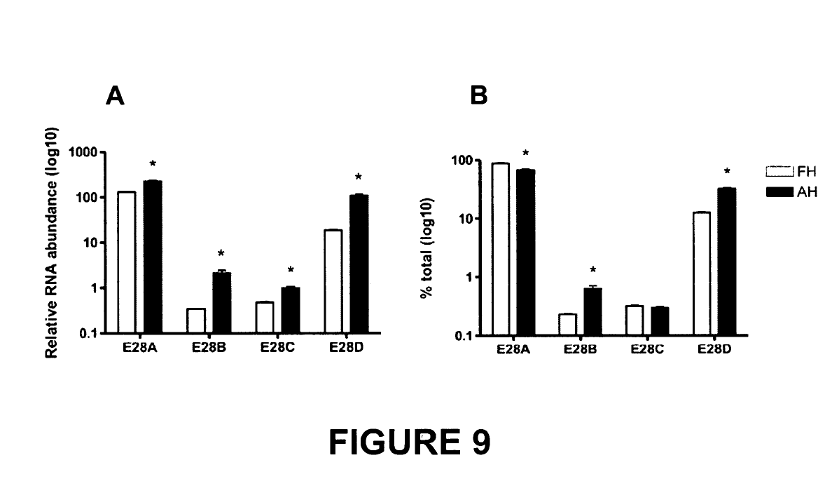

Splice variants of Naᵥ1.5 in C-terminus are known to vary during development. Therefore, we investigated whether our novel 3′ isoforms showed similar behavior. Quantitative real time RT-PCR indicated that the relative abundances of each of the isoforms increased by 41.6% (p<0.001), 5.1 fold (p<0.01), 1.1 fold (p<0.01) and 4.8 fold (p<0.001) for E28A, E27B, E28C, and E28D from fetal to adult heart, respectively (FIG. 9A). FIG. 9B shows that as a percentage of the total transcripts, E28A was the most abundant in both fetal and adult heart. E28B was the least abundant in fetal heart but increased the most with development, changing 1.7±0.2 fold. As a percentage of the total mRNA, the alternative splice variants E28B and E28D increased significantly, the full length E28A decreased, and the E28C abundance was unchanged during development.

Heart Failure Increased Two of the Sodium Channel C-Terminal Splice Variants.

The presence of splice variants was compared between the ventricles obtained from 12 patients whose hearts were removed during cardiac transplantation for HF (Table 1), and three control patients with no known cardiac disease.

TABLE 1

| Patients # | Type of CM | Gender | Age |
|---|---|---|---|
| 1 | DCM | M | 60 |
| 2 | DCM | M | 54 |
| 3 | DCM | M | 62 |
| 4 | ICM | M | 43 |
| 5 | DCM | M | 45 |
| 6 | DCM | M | 51 |
| 7 | DCM | F | 59 |
| 8 | DCM | M | 63 |
| 9 | DCM | M | 60 |
| 10 | DCM | M | 45 |
| 11 | DCM | M | 63 |
| 12 | ICM | M | 47 |

DCM: dilated cardiomyopathy
ICM: ischemic cardiomyopathy
CM: cardiomyopathy
M: male
F: female

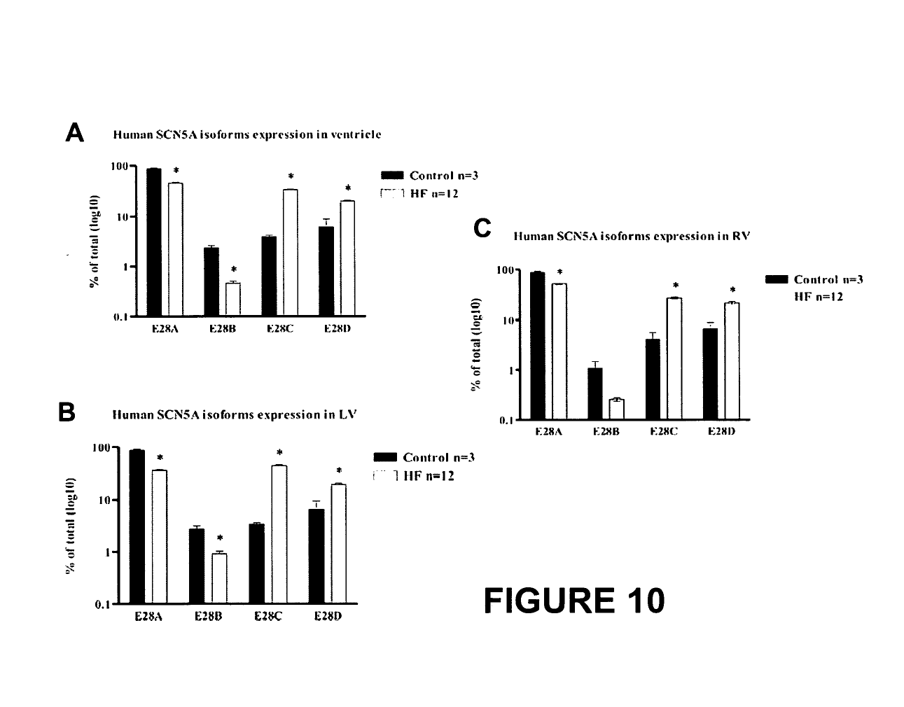

Real time RT-PCR results indicated that the relative mRNA abundance of E28A full length isoform was decreased by 24.7% in HF patients compared to controls (p<0.001). Two of the truncated isoforms were increased in HF patients as compared to the controls (FIG. 10A), however. The E28C and E28D mRNA abundances were increased 14.2 fold (p<0.001) and 3.8 fold (p<0.001) respectively comparing controls to HF patients (FIG. 10A). On the other hand, the least abundant isoform, E28B, decreased 73.8% (p<0.01) in HF patients. As a percentage of the total transcript abundance, E28A and B decreased significantly from 87.5% (±5.1) and 2.4% (±0.4) in controls to 45.1% (±4.5) and 0.5% (±0.2) in HF patients. The E28C and D variants increased from 3.9% (±0.6) and 6.2% (±4.6) in controls to 34.3% (±3.1) and 20.2% (±3.3) in HF patients (FIG. 10A). The total percentage of short isoform variants went from 12.5% (±5.1) of the total SCN5A mRNA in control subjects to 54.9% (±4.5) in HF patients.

Because inhomogeneities of channel expression are thought to contribute to arrhythmic risk, the relative RNA abundance of the SCN5A isoforms were compared in the left (LV, FIG. 10B) and right ventricles (RV, FIG. 10C) of controls and HF patients. Normalized to the total SCN5A mRNA, E28A abundances were decreased in both the LV and RV. The pattern of changes for the truncation variants was similar in both ventricles with increases in E28C and E28D. The percentage of truncated mRNAs was increased more in the LV when compared to the RV (p=0.0003).

Tissue-Specific SCN5A Splicing.

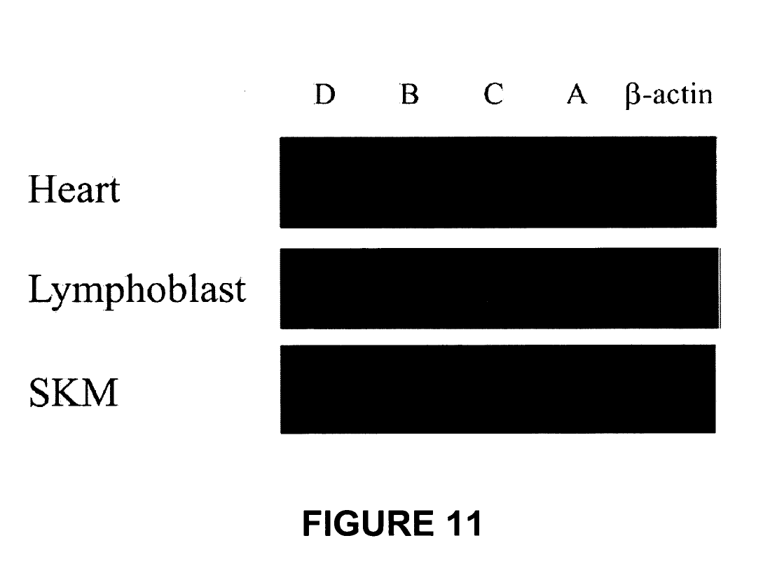

In addition to heart, cardiac Sodium channel mRNA is known to be transcribed in skeletal muscle and leukocytes. The presence of the novel C-terminal splice variants was assessed in these tissues. Real time RT-PCR result showed that only two of the four splice variants were transcribed in skeletal muscle, but all four isoforms were detected in the human lymphoblast (FIG. 11). The distribution of those four isoforms in lymphoblasts was different than that in heart and was 22.3% (±4.2), 12.6% (±1.7), 50.8% (±8.2) and 14.4% (±3.1) for E28A, E28B, E28C, and E28D, respectively, in lymphoblasts from patients (n=4) with no cardiac dysfunction.

Truncated Isoforms Reduce Sodium Channel Function.

The three novel SCN5A splice variations identified were predicted to result in prematurely truncated, nonfunctional Sodium channels. We tested this idea by making a gene-targeted mouse model with a nonsense mutation in exon 28 (1652stop, FIG. 7). Proper homologous recombination in embryonic stems cells was confirmed by sequencing, restriction site analysis, and Southern blotting (see supplemental material). Embryonic stem (ES) cells were successfully injected into blastocyst and produced chimeric animals. Nevertheless, this mutation was lethal to embryos when attempting to breed these animals. Undifferentiated mouse ES cells heterozygous for the SCN5A^{1652stop} had normal growth characteristics and could be differentiated into spontaneously beating cardiomyocytes (CMs), however.

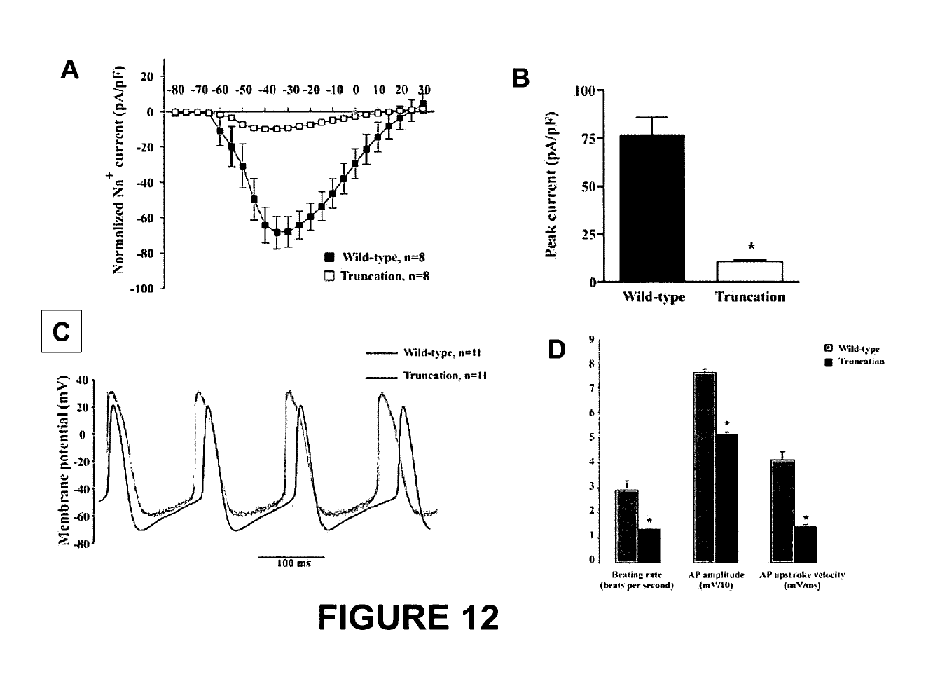

To assess the electrophysiological effect of the presence of the truncation, ES cells heterozygous for the mutation were differentiated in vitro to CMs and studied electrophysiologically. Current and action potentials (APs) were compared from single CMs enzymatically isolated at day 19. The Sodium channel current-voltage relationships from contracting CMs isolated from wild-type and truncation embryonic bodies derived from the respective ES cell lines are shown in FIG. 5A. The peak I_{Na} was decreased by 86.1% (±5.2, n=8, p=0.0002) in differentiated CMs containing the truncation when compared to that of WT (76.4±9.5 pA/pF to 10.6±0.9 pA/pF; FIG. 5B). Action potentials recorded in the current clamp mode from spontaneously beating CMs showed significant slowing of the beating frequency from 2.9±0.4 beats per second (bps) in the wild-type to 1.3 bps±0.1 (p=0.02, n=11) in the truncation mutant. Action potentials also showed a significant reduction in the maximum rate of rise of the AP in the truncation mutation from 4.1±0.3 mV/ms to 1.4±0.1 mV/ms (p<0.01, n=11) and a reduced amplitude from 76±1.4 mV to 52±0.6 mV (p≦0.01, n=11) in comparison with wild-type (WT) (FIGS. 12C and D). These changes are consistent with reduced sodium channel function.

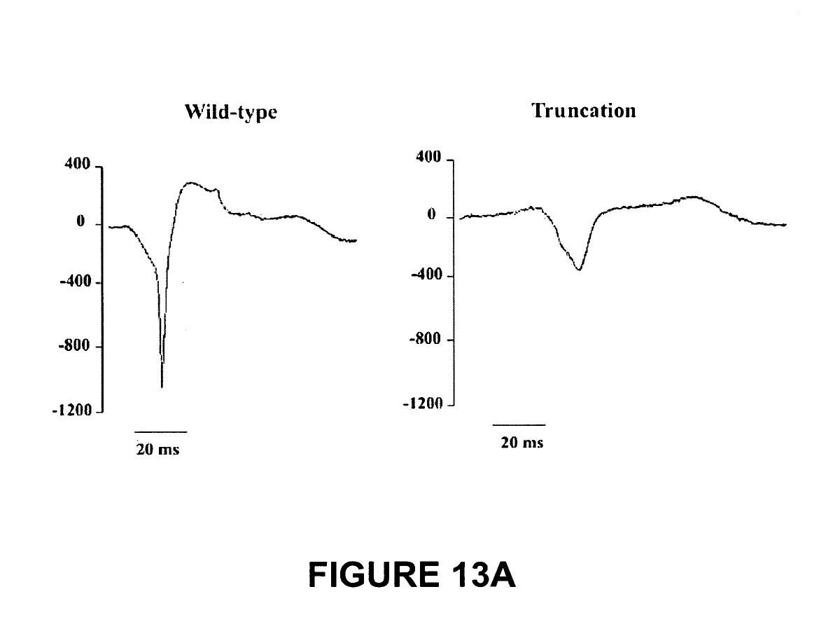

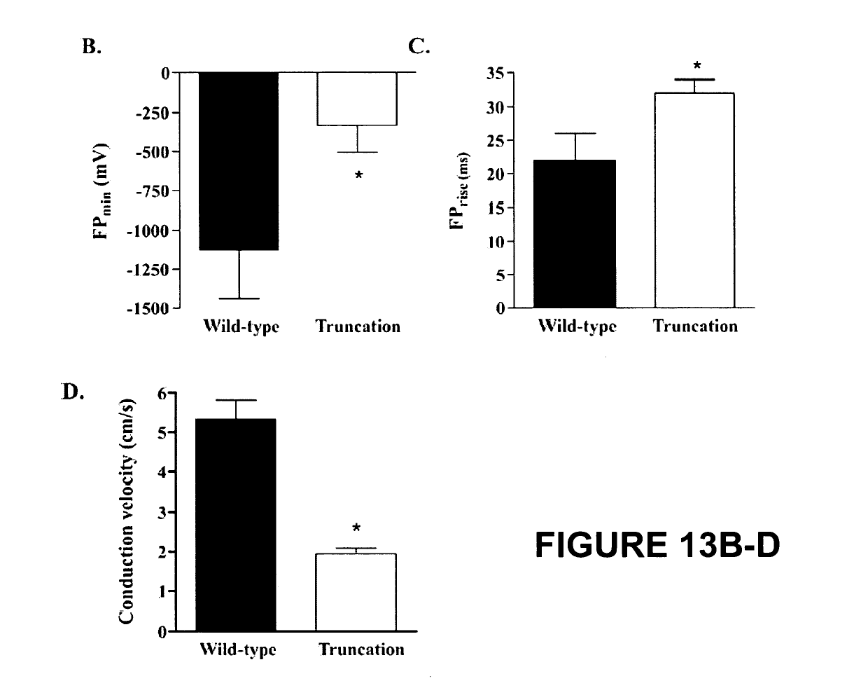

Syncytial properties of CMs containing the truncation mutation were studied by dissecting areas of CMs from embryoid bodies and placing them on top of planar multi-electrode arrays (MEAs) (Caspi, and Gepstein. 2004. Potential applications of human embryonic stem cell-derived cardiomyocytes. Ann. N.Y. Acad. Sci. 1015:285-298; and Kehat et al. 2002. High-resolution electrophysiological assessment of human embryonic stem cell-derived cardiomyocytes: a novel in vitro model for the study of conduction. Circ. Res. 91:659-661). Characteristics of bipolar extracellular electrograms from WT and mutated ES cells were compared. The time of the initial decline of the bipolar field potential (FP), the minimum FP amplitude, and conduction velocity are known to reflect sodium channel activity. Consistent with a physiologically significant reduction in sodium current as a result of the truncated mRNA, MEA recordings of CMs with the truncation mutation showed the minimum FP decreased by 70.5% (p<0.05) from −1126±314 μV (n=6) in WT to −332±174 μV (n=7) in the truncation (FIGS. 13A and B). The FP rise slowed by 45.5% (p<0.05) from 22±4 ms (n=6) to 32±2 ms (n=7) in WT and the mutant (FIGS. 13A and C), respectively. The conduction velocity was decreased by 64.2% (p=0.03) from 5.3±1.2 cm/s (n=6) in WT to 1.9±0.4 cm/s (n=7) in the truncation (FIG. 13D).

Discussion

New cardiac sodium channel C-terminal splicing variants were reported. Each of these variants is expected to result in prematurely truncated, nonfunctional sodium channels. These variants are differentially regulated during development. As compared to controls, patients with HF showed an increase in the total truncated forms. A mouse model of the splice variants confirmed that truncated channels failed to produce sodium current and had adverse effects on myocyte electrophysiology. Splicing variations were seen also in lymphoblasts and skeletal muscle, which are known to express cardiac sodium channels.

Alternative C-terminal splicing seems to be a common biological motif. C-terminal exon switching with alternative polyadenylation has been found in a number of ion genes or their receptors, such as P2X receptors (Koshimizu et al. 2006. Carboxyl-terminal splicing enhances physical interactions between the cytoplasmic tails of purinergic P2X receptors. Mol. Pharmacol. 69:1588-1598), α-subunits of large conductance calcium- and voltage-activated potassium channels (Chen et al. 2005. Functionally diverse complement of large conductance calcium- and voltage-activated potassium channel (BK) α-subunits generated from a single site of splicing. J. Biol. Chem. 280:33599-33609), P/Q-type Ca²⁺ channels (Chaudhuri et al. 2004. Alternative splicing as a molecular switch for Ca²⁺/calmodulin-dependent facilitation of P/Q-type Ca²⁺ channels. J. Neurosci. 24:6334-6342), KCNQ potassium channels (Ohya et al. 2003. Molecular variants of KCNQ channels expressed in murine portal vein myocytes: a role in delayed rectifier current. Circ. Res. 92:1016-1023), the voltage-dependent calcium channel α_{1A}-subunit (Zhuchenko et al. 1997. Autosomal dominant cerebellar ataxia (SCA6) associated with small polyglutamine expansions in the α1A-voltage-dependent calcium channel. Nat. Genet. 15:62-69; and Krovetz et al. 2000. C-Terminal alternative splicing changes the gating properties of a human spinal cord calcium channel α1A subunit. J. Neurosci. 20:7564-7570), and the K_{ATP} channel (K_{IR}6.x) (Shi et al. 2005. Function and distribution of the SUR isoforms and splice variants. J. Mol. Cell Cardiol. 39:51-60). It is thought that splice variation allows a mechanism to fine tune the behavior of the channels. Nevertheless, in the case of the cardiac sodium channel, it is hard to understand the biological benefit of this alternatively splicing since it seems to generate only inactive channels, and reduced sodium channel function is thought to be arrhythmogenic (George. 2005. Inherited disorders of voltage-gated sodium channels. J. Clin. Invest 115:1990-1999; and Antzelevitch et al. 1999. Transmural dispersion of repolarization and arrhythmogenicity: the Brugada syndrome versus the long QT syndrome. J. Electrocardiol. 32 Suppl:158-165).

The variant mRNAs described are expected to result in premature, nonfunctional channels that resemble reported Brugada syndrome mutations. Brugada syndrome is an inherited form of sudden death that is caused by a reduction in cardiac Na⁺ current. Brugada mutations likely to be similar in effect to the splice variants observed here include frameshift errors, splice site defects, and premature stop codons.

We directly tested the physiological effects of premature truncation of the sodium channel in a gene-targeted mouse model. In this model, introducing a premature stop codon at 1652 into one of two alleles resulted in an 86% reduction in Na⁺ current. This dominant negative effect was unexpected but has been seen with the cardiac sodium channel (Makielski et al. 2003. A ubiquitous splice variant and a common polymorphism affect heterologous expression of recombinant human SCN5A heart sodium channels. Circ. Res. 93:821-828) and other ion channels. Therefore, this result suggests that, at the levels seen in this study, the truncation variants could result in electrophysiological abnormalities and arrhythmic risk, perhaps not as severe as seen in the heterozygous mouse model but sufficient to cause a forme fruste of Brugada Syndrome.

Heart failure (HF) is known to be associated with increased arrhythmic risk and reduced Na⁺ current. In this study, we found that patients with severe forms of HF had increased levels of abnormally truncated SCN5A mRNA and reduced full length mRNA, especially in the UTR, possibly explaining these observations. Brugada patients with SCN5A mutations likely to give rise to similar electrophysiological effects are at increased arrhythmic risk, and our mouse model showed severe abnormalities in sodium channel-dependent parameters. Therefore, it seems possible that at the levels observed in the HF patients, the effects of the combination of reduced full length transcript abundance and increases in the abundances of the abnormal splice variants may contribute to the arrhythmic risk in HF patients. The reduced amount of abnormal mRNA in our case as compared to Brugada syndrome and the predilection for changes to be most prominent in the left heart may explain why HF patients did not show the classic baseline electrocardiographic changes most prominent in the right surface electrocardiographic leads of Brugada syndrome patients.

C-terminal splicing isoforms were seen in all tissues known to transcribe SCN5A. Nevertheless, the number of isoforms and their relative abundances varied between these cell types. Skeletal muscle showed only two isoforms, while lymphoblast showed all four.

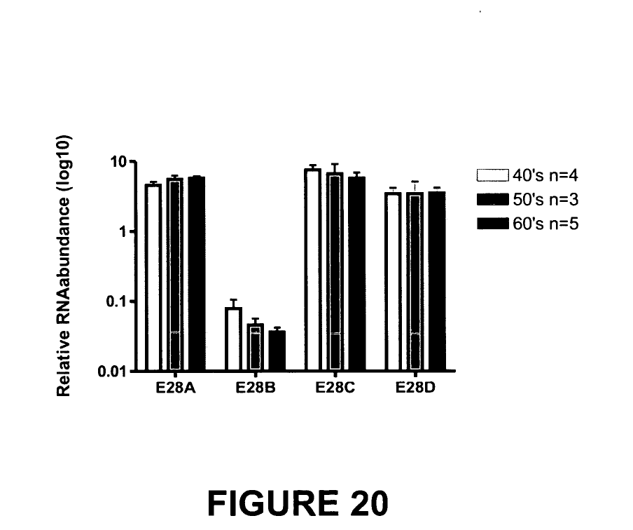

There are several limitations to our study. First, the truncation mutation that we made did not result in exactly the same premature truncation as any of the four novel splice variations. Our mouse model was most similar to the expected effect of E28D, resulting in a premature truncation eight amino acids away. Therefore, the electrophysiological effects of the naturally occurring splice variations may differ somewhat from our mouse model, but in any case, a reduction in sodium current is likely. Also, in this study, most RNA samples were from males with dilated cardiomyopathy. Similar splice variations were seen in the single female and two ischemic cardiomyopathy (ICM) samples, but gender and pathology-specific differences may exist that were not uncovered here. Finally, while the control subjects were younger than the HF patients, we could find no evidence of splice variation changes with age (FIG. 20).

In conclusion, we demonstrate that there are several alternatively truncated forms of SCN5A mRNA in human hearts. During HF, these alternatively spliced mRNA isoforms are likely to reduce sodium current to levels that might contribute to arrhythmic risk alone or in combination with other inciting causes.

### EXAMPLE 3

Methods

Cell Culture and Cell Viability Assay

The rat embryonic cardiomyocyte cell line, H9c2 (ATCC cat # CRL-1446), or acutely isolated neonatal rat heart cardiomyocytes were used. Rat neonatal ventricular cells were isolated from 3-day-old Sprague-Dawley rats (Charles River Laboratories Wilmington, Mass.) using a kit and following the manufacturer's instructions (Worthington Biochemical Corp. Lakewood, N.J.). Cells cultured in Dulbecco's modified Eagle's medium (DMEM; ATCC, Manassas, Va.) with 10% fetal calf serum (ATCC) under standard tissue culture conditions at 37° C. to 70-80% confluence were exposed to AngII (Sigma, St. Louis, Mo.) or H2O2 (Sigma) in serum free culture medium (SFM) for a total of 48 h in triplicate in 24 well plates. Experiments were repeated three times. After dissociation with 0.125% trypsin-EDTA, 20 μL of 0.4% Trypan-blue (Sigma, St. Louis, Mo.) was added to each well, and a Trypan-blue exclusion viability assay was performed. The use of rats conforms with the Guide for the Care and Use of Laboratory Animals published by the US National Institutes of Health (NIH Publication No. 85-23, revised 1996).

Quantification of scn5a Transcripts by Quantitative Real-Time RT-PCR Assay

To determine the abundance of cardiac sodium channel (scn5a) mRNA under the various conditions, quantitative real-time RT-PCR was used. Total RNA from untreated and treated cardiomyocytes was isolated using the RNeasy Mini Kit (Qiagen, Valencia, Calif.) with the addition of RNase-free DNase I. Reverse transcription was carried out at 42° C. for 30 min with iScript reverse transcriptase (Bio-Rad, Hercules, Calif.), 1 pg total RNA, and 4 μL of 5× iScript reaction mix following the manufacturer's instructions. The first strand cDNA was used as Green Supermix (Bio-Rad) and 2.5 μM primer pairs in total 25 μL reaction volume. The forward primer rtPCRscn5aF (5′ GAAGAAGCTGGGCTCCAAGA 3′ (SEQ ID NO. 28)) recognized a sequence from exon 26. The reverse primer, rtPCRscn5aR (5′ CATCGAAGGCCTGCTTGGTC 3′ (SEQ ID NO. 29)), was complementary to exon 27 of scn5a cDNA. The reactions gave rise to a 101 bp PCR product. All amplifications were performed in triplicate and consisted of 40 cycles of 30 s at 95° C., 30 s at 60° C., and 30 s at 72° C. in a BioRad thermocycler icycler (Hercules, Calif.). PCR products were analyzed by relative standard curve methods. β-Actin was used as a reference when making quantitative comparison.

Electrophysiological Determination of Na+ Current

Three hours prior to the start of the patch-clamp experiments, H9c2 cells were trypsinized and plated on treated plastic coverslips. H9c2 cells were treated with or without H₂O₂ as indicated. Glass pipettes were pulled on a Sutter Model P-97 horizontal puller to a resistance of 0.5 to 1.5 MΩ. The glass pipettes were filled with a solution of (in mM) CsCl 60, Cesium Aspartate 80, EGTA sodium 11, HEPES 10, Na₂ATP 5 and pH 7.2 with CsOH. The bath solution consisted of (in mM) Na 30, N-methyl-D-glutamate chloride 100, CsCl 5, CaCl₂ 2, MgCl_{2 1.2,} HEPES 10, Glucose 5 and pH 7.4 with NaOH. Once a seal was established, a small amount of suction was applied to obtain the whole cell configuration. From a holding potential of −100 mV, peak currents obtained at −10 mV were used for comparison. Cells were tested at 25° C. Data were sampled at 10 kHz and later filtered at 5 kHz for analysis. Currents were recorded and analyzed with an Axopatch 200B amplifier, Axon Digidata 1230A A/D converter and pClamp software (Molecular Devices Corporation, Sunnyvale, Calif.).

Promoter-Reporter Constructs and Transient Transfection

The scn5a promoter region has previously been defined (Shang, L. L. & Dudley, S. C., Jr. (2005) J. Biol. Chem. 280, 933-940). For these experiments, a new promoter construct that contained the NF-κB consensus binding site was used to test the effect of treatments on scn5a transcription. This construct, pGL3-APS3, consisted of a 937 bp fragment starting from exon 1C to +32 base pairs relative to the start codon located on exon 2 of mouse scn5a gene.

H9c2 cardiomyocytes were plated in each well of 24-well plates at a density of 2.5×10₄ cells in a final volume of 1 mL of culture medium, allowed to attach overnight, and expand to 70%-80% confluence. Transfection of 0.3 μg of the promoter-reporter construct and 0.013 μg of a plasmid containing the herpes simplex virus thymidine kinase (HSV-TK) promoter driving expression of a synthetic Renilla luciferase (phRL-TK; Promega, Madison, Calif.) was carried out with 0.9 μL of Fugene6 chemical transfection reagents (Roche, Indianapolis, Ind.) following the manufacturer's instructions. The serum free DMEM cultural media with or without AngII or H₂O₂ was changed every 24 h. After culture for 48 h, the cells were treated with passive lysis buffer (Promega, Madison, Calif.), and cell extracts were collected for analysis of firefly and Renilla luciferase activities using 100 μL of luciferase assay substrate and 100 μL of Stop & Glo reagent of the dual-luciferase reporter assay system (Promega, Madison, Calif.). Light emission was quantified in a Veritas microplate luminometer using Veritas-version 1.4.0 software (Tuener Biosystems, Sunnyvale, Calif.). Transfection efficiency of the reporter constructs was controlled by comparison to Renilla luciferase activity. The phRL-TK vector minimized any modulation of Renilla luciferase expression by the experimental conditions since it has been engineered to remove the majority of potential transcription factor binding sites. The luciferase activity of the all promoter-constructs was normalized to a pGL3-basic promoter-less control transfected simultaneously. Four separate transfection sessions were analyzed, and at each session, transfections were performed in triplicate. Three dual luciferase readings were taken for each transfection experiment.

Site-Directed Mutagenesis of NF-κB Binding Site

Disruption of the NF-κB binding site was undertaken using the QuikChange II XL sitedirected mutagenesis kit according to the manufacturer's instructions (Stratagene, La Jolla, Calif.). Briefly, for PCR 10 ng of pGL3-APS3 was used as a template, and the nucleotide primers listed were used to mutate the NF-κB binding site (the bold as wild type, the underline as mutant) of pGL3-APS3: NFκB-mutCF: 5′GGTGCTGCACTCAGGCCATCCCTATGAGATCCTC 3′ (SEQ ID NO. 30) and NFκB-mutCR: 5′ GAGGATCTCATAGGGATGGCCTGAGTGCAGCACC 3′ (SEQ ID NO. 31). After digestion with DpnI, 2 μL of PCR product were used to transform XL10-Gold competent cells. Sequencing identified appropriate clones.

Electrophoretic Mobility Shift Assay (EMSA/Gel-Shift)

The H9c2 cells were treated for 48 h with AngII or H₂O₂, with or without CAPE (caffeic acid phenethyl ester, an NF-κB inhibitor at 10 μM) starting 24 h after plating. Approximately 5×10⁶ cells were scraped for nuclear protein extraction by nuclear extract kit (Activemotif, Carlsbad, Calif.). A double-stranded oligonucleotide containing the consensus-binding sequence (bold) for NF-κB (5′GGTGCTGCACTCAGGGGATCCCTATGAGATCCTC 3′ (SEQ ID NO. 32)) and NF-Kb mutant sequence (5′GGTGCTGCACTCAGGCCATCCCTATGAGATCCTC 3′ (SEQ ID NO. 33)) from scn5a promoter were used as probes to assay for binding activity of the nuclear extracts. Protein-DNA complexes were detected using biotin end-labeled double-stranded DNA probes prepared by annealing complementary oligonucleotides. Oligonucleotides were labeled in a reaction using terminal deoxynucleotide transferase and biotin-N4-CTP (Pierce, Rockford, Ill.) following the biotin 3′ end DNA labeling kit manual. The binding reaction was performed using the LightShift kit (Pierce). Briefly, 30 μg of nuclear extracts and binding buffer were incubated on ice for 5 min in a volume of 20 μL, then the labeled probe (20 fmol) was added, and the reaction was allowed to incubate for an additional 25 min. Following electrophoresis, the DNA-protein complexes were transferred onto nylon membranes and detected using chemiluminescence. TNF-α activated H9c2 cell nuclear extract (5 μg) was used as positive control. The reaction products were separated on a 6% retardation gel. Specificity was confirmed by addition of unlabelled probe in 200-fold excess.

Chromatin Immunoprecipitation (ChIP) Assay

Formaldehyde cross-linking and chromatin immunoprecipitation was performed as described in manufactory's manual (ChIP-IT™ kit, Activemotif). Briefly, proteins were crosslinked with chromatin using 1% formaldehyde in H9c2 cells with or without treatment. The cells were subsequently sonicated in lysis buffer, and an aliquot of the lysate was used in a PCR reaction. The remaining lysate was cleared with protein G beads. One half of the cleared lysate was incubated with p50 or p65 antibody, while the other half was used as a negative control without the antibody. After reversing the cross-linking, the immuno-complex was digested with proteinase K, and the DNA was purified. DNA was analyzed by PCR with the PicoMax Polymerase (Stratagene, La Jolla, Calif.) and primers specific to the APS3 promoter region.

Stable H9c2 Cell Lines Overexpressed NF-κB Subunits p50 and p65

The H9c2 cells were co-transfected with expression vectors carrying human NF-κB subunits p50 and/or p65 (Lindholm et al. (2003) J. Hypertens. 21, 1563-1574) and pDsRed-express-N1 vector carrying red fluorescent protein as marker (Clontech, Mountain View, Calif.) and selected with 400 μg/mL geneticin (Invitrogen) for at least for four weeks. At which time, over 90% of the cells showed red fluorescence. Transfection was confirmed by RT-PCR using human p50 or p65 specific primers. The SYBR quantitative real-time RT-PCR was used to assay the Na+ channel expression.

Statistical Evaluations

All data are present as means±S.E.M. Statistical analysis of mean values was carried out using Student's paired or unpaired t tests. ANOVA was used for comparison of variance between multiple means. A p value<0.05 was considered statistically significant.

Results

AngII and H₂O₂ Dose Ranging in H9c2 Cells

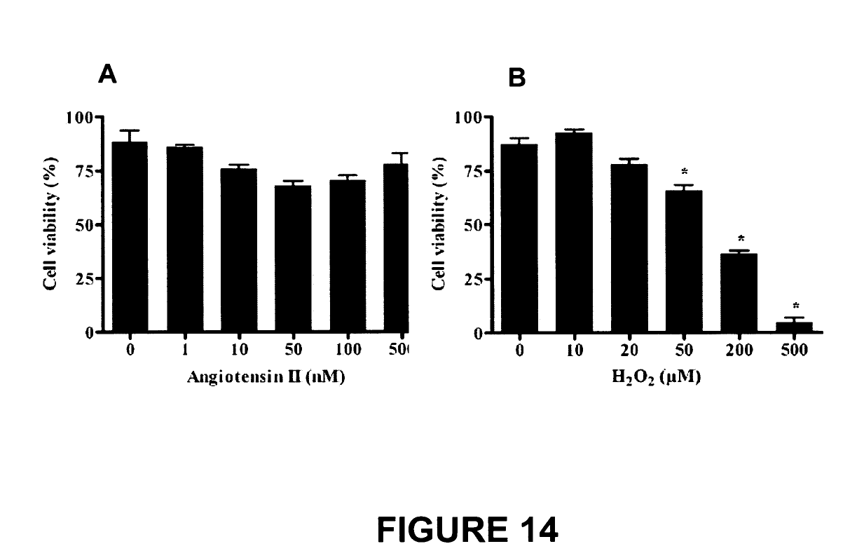

To determine appropriate concentrations of these agents in future experiments, rat H9c2 cardiomyocytes were treated with escalating concentrations of AngII and H₂O₂, and the dose-dependent cell viability was determined. H9c2 cardiomyocytes were tolerant of a wide range of AngII concentrations from 1-500 nM in serum free medium (FIG. 14A). On the other hand, higher doses of H₂O₂ induced cell death starting at concentrations of 50 μM (FIG. 14B). Therefore, we restricted further experiments to 20 μM H₂O₂, where there was no statistically significant increase in cell death over the time course of our experiments. Exposures of 48 h were used to allow sufficient time for transcriptional effects on the Na⁺ current.

Scn5a mRNA Abundance was Downregulated by AngII

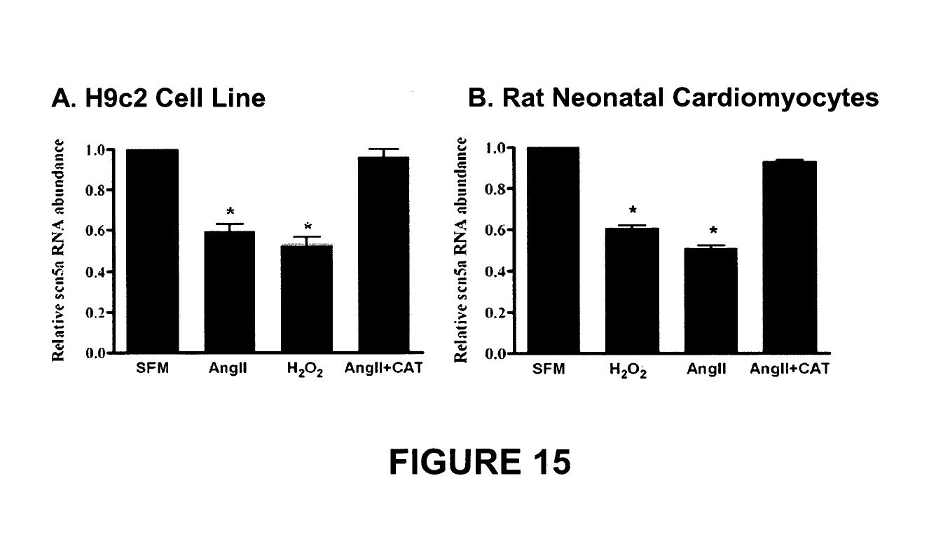

H9c2 cells treated with 100 nM AngII for 48 h showed a 47.3% (±5.3%, n=7, P<0.01) reduction in scn5a mRNA abundance (FIG. 15A). Because H9c2 cells are generally responsive only to high doses of AngII (Tran et al. (1995) Biochim. Biophys. Acta 1259, 283-290; and Laufs et al. (2002) Cardiovasc. Res. 53, 911-920), experiments were repeated with acutely isolated rat neonatal cells and a more physiological concentration of AngII. In neonatal cardiomyocytes, a 48 h exposure to 2 nM AngII resulted in a 49.0% (±5.2%, n=10, P<0.01) reduction in scn5a mRNA abundance in neonatal cardiomyocytes, suggesting that isolated myocytes were more sensitive and the effect was not limited to the H9c2 line (FIG. 15B).

Both Cardiac Na⁺ Channel mRNA and Current were Downregulated by H₂O₂

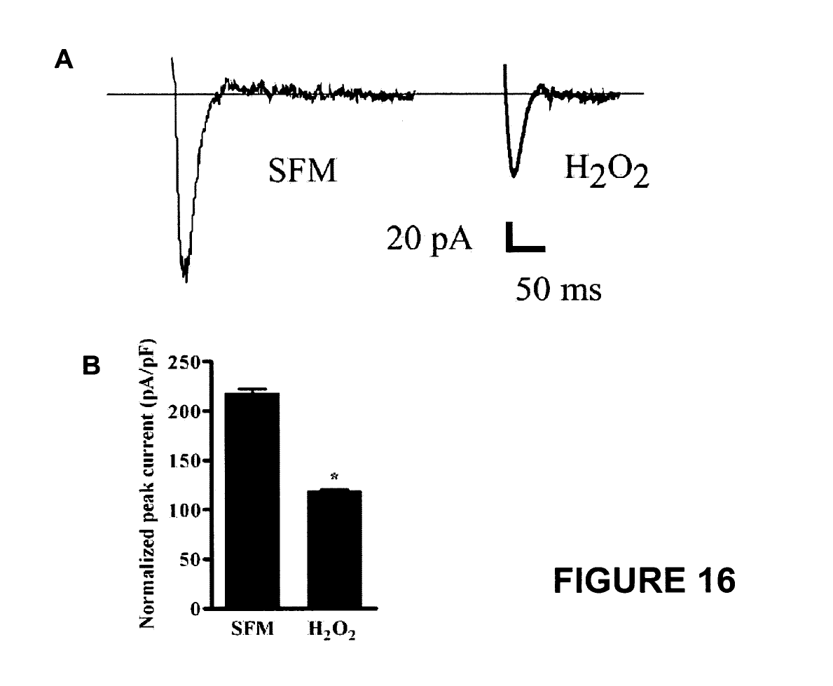

Because AngII is known to activate the NADPH oxidase generating superoxide that is dismutated to H₂O₂, we tested whether the H₂O₂ would recapitulate the AngII results. Exposure of H9c2 cells to 20 μmol/L H₂O₂ for 48 h caused a similar reduction in Na⁺ channel mRNA (46.9%±3.6%, n=9, p<0.01; FIG. 15A). In isolated neonatal rat myocytes and consistent with their increased sensitivity to AngII, there was an analogous response to a reduced concentration of H₂O₂ for 48 h (40 nM; 45.4%±7.5%, n=9, p<0.01; FIG. 15B). In both types of myocytes, the AngII-mediated downregulation of Na⁺ channel mRNA could be prevented by pretreatment of the cells with catalase, consistent with the idea that AngII was acting through H₂O₂ production. FIG. 16 shows that 20 μmol/L H₂O₂ exposure for 48 h resulted in a 46.0% ( ±2.9%) decrease in Na⁺ current commensurate with the reduction in mRNA abundance (n=7 for control, n=9 in treated group, P=0.01).

Evidence of NF-κB Regulation of the Cardiac Na⁺ Channel

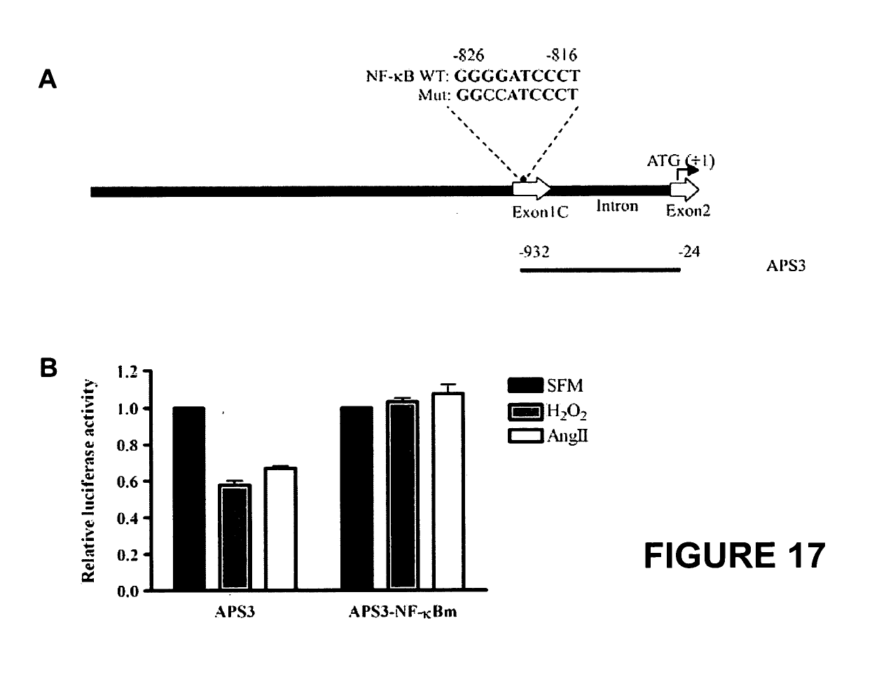

NF-κB is a known redox sensitive transcription factor (Zhou et al. (2001) Free Radic. Biol. Med. 31, 1405-1416). Previously, we have shown that the promoter region of the cardiac Na⁺ channel contains one NF-κB consensus binding sequence (Shang, L. L. & Dudley, S. C., Jr. (2005) J. Biol. Chem. 280, 933-940). Therefore, we investigated whether NF-κB might be involved in the AngII or H₂O₂-mediated Na⁺ channel transcriptional regulation. To test this idea, we constructed a mutated form of the scn5a promoter in which the NF-κB binding site had been altered, APS3-NF-κBm (FIG. 17A). Promoter-reporter constructs containing the intact NF-κB binding site showed reductions in activity in response to AngII or H₂O₂ (FIG. 17B). The scn5a Na⁺ channel promoter activity was depressed by 33.0% (±2.6%, n=4, P<0.001) and 42.3% (±4.5%, n=4, P<0.001), respectively, in H9c2 cells when cardiomyocytes transfected with the APS3 construct were compared with and without AngII or H₂O₂ exposures. On the other hand, the construct with a mutated NF-κB binding site showed no significant change in activity in the presence of AngII or H₂O₂.

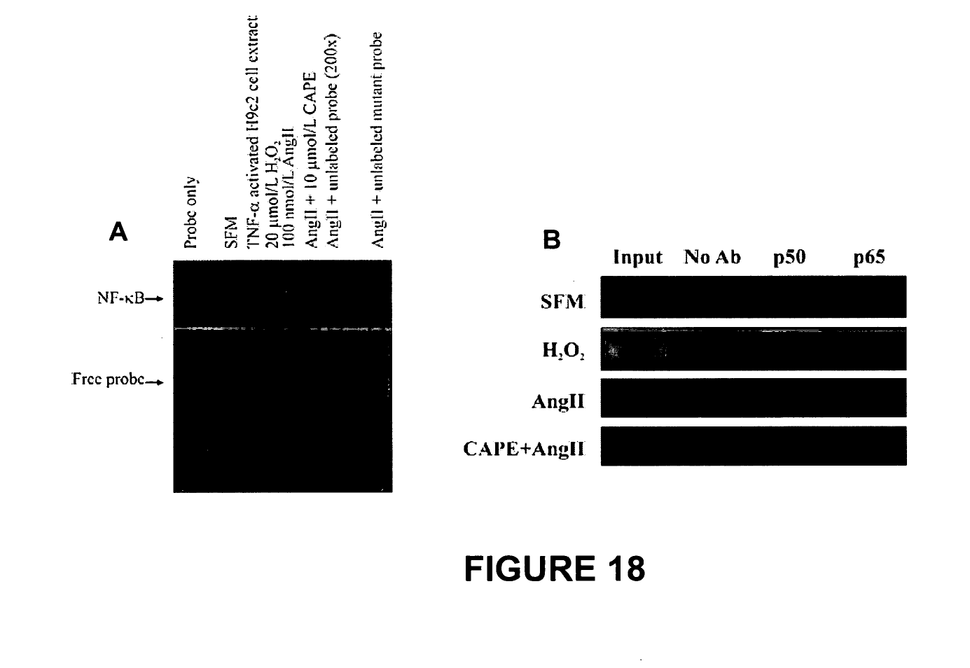

To confirm that NF-κB was binding to the scn5a promoter in response to AngII or H₂O₂ exposure, we employed electrophoretic mobility shift and chromatin immunoprecipitation (ChIP) assays. A 35 bp fragment of the scn5a promoter containing the NF-κB site was used as the probe (FIG. 18A). The NF-κB binding activity increased in the presence of AngII or H₂O₂ treatment. NF-κB binding was blocked by caffeic acid phenethyl ester, an NF-κB inhibitor (Natarajan et al. (1996) Proc. Natl. Acad. Sci. U.SA 93, 9090-9095; Watabe et al. (2004) J. Biol. Chem. 279, 6017-6026). The ChIP assay showed that there was formation of the complete p50-p65 NF-κB heterodimer on the cardiac Na⁺ channel promoter in response to AngII or H₂O₂ treatment (FIG. 18B).

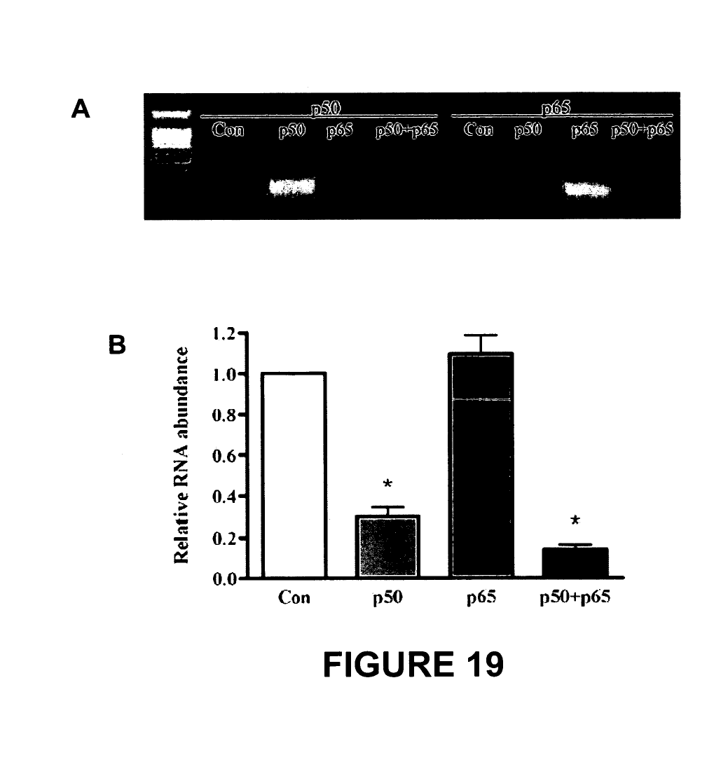

Overexpression of NF-κB subunits in H9c2 cells confirmed the necessity of the p50 subunit for Na⁺ channel downregulation. Quantitative real-time RT-PCR result showed that the relative scn5a mRNA abundances were decreased in cell lines expressing p50 only or the combination of p50 and p65 by 77.3% (±7.3, n=4) and 88.6% (±4.8, n=4), respectively. There was no significantly change in Na⁺ channel mRNA in the presence of p65 overexpression alone, however (FIG. 19).

Discussion

An appropriate number and function of voltage-gated cardiac Na⁺ channels (scn5a) are critical for normal cardiac electrical activity. Either excessive or reduced channel current increases arrhythmic risk. Downregulation of the cardiac Na⁺ channel is seen in heart failure, a condition associated with increased RAS activation (Valdivia et al. (2005) J. Mol. Cell Cardiol. 38, 475-483), and this reduction may contribute to the arrhythmic risk seen in this condition. Recently, it has been shown that cardiac-restricted ACE overexpression mice, which have elevated local AngII levels, show conduction blocks, increased risk for ventricular arrhythmias, and increased sudden death rates, consistent with Na⁺ channel downregulation (Kasi et al. (2005) Circulation 112, Suppl. 17, 11-248). In this model, scn5a mRNA abundance is reduced. Therefore, we hypothesized that RAS activation might result in the scn5a transcriptional downregulation.

Our data imply that AngII can regulate scn5a transcription over a 48 h period, this effect is mediated by H₂O₂, and this effect is significant in terms of Na+channel current. Cardiomyocytes exposed to AngII showed a reduction in scn5a mRNA abundance. Compatible with the idea that AngII stimulates reactive oxygen species production by NAD(P)H oxidases (Griendling et al. (1994) Circ. Res. 74, 1141-1148), the AngII effect was similar to that of H₂O₂ and was blocked by loading cells with catalase prior to AngII exposure. Moreover, H₂O₂ resulted in a reduction in Na⁺ channel current corresponding to the reduction in mRNA abundance, implying that transcriptional regulation may play an important role in Na⁺ current availability over the 48 h period.

Our data suggest that NF-κB mediates the effects of AngII and H₂O₂. NF-κB is a redox sensitive transcription factor, and both the human and mouse Na⁺ channel promoters have one NF-κB consensus binding site (Genbank accession numbers AY313163 and AY769981). AngII and H₂O₂ are known to activate NF-κB in cardiomyocytes (Watabe et al. (2004) J. Biol. Chem. 279, 6017-6026; Rouet-Benzineb et al. (2000) J Mol Cell Cardiol 32, 1767-1778; Brasier et al. (2000) Mol Cell Biochem 212, 155-169; and Frantz et al. (2001) J Biol Chem 276, 5197-5203). Our data show that: 1) NF-κB binds to the channel promoter during treatment with either agent, 2) mutation of the NF-κB binding site prevents the effects of either agent on promoter activity, and 3) overexpression of NF-κB recapitulates the effects of either agent. Based on the ChIP assay and NF-κB subunit overexpression, Na⁺ channel downregulation seems to be mediated by p50 subunit binding to the promoter.

Our data suggest that arrhythmic risk seen in states of RAS activation or oxidative stress might be, in part, the result of Na⁺ channel transcriptional downregulation. This chronic effect may be additive with previous reported acute effects. In one report, Na⁺ current was inhibited when the cardiac cells were exposed to tert-butylhydroxyperoxide (Bhatnagar et al. (1990) Circ. Res. 67, 535-549). Others have reported that H₂O₂ exposure results in a slowing of inactivation of the Na⁺ current, an effect dependent on activation of PKC (Ward et al. (1997) J. Physiol 500 (Pt 3), 631-642). Therefore, it seems likely that multiple acute and chronic deleterious effects on Na⁺ channels can contribute to the observed arrhythmic risk associated with oxidative stress and RAS activation.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. An isolated polynucleotide having a nucleic acid sequence of E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), E28D (SEQ ID NO. 9), or complements thereof.

2. A method for detecting, diagnosing, or prognosticating an increased risk of heart disease in an individual, the method comprising the step of detecting the presence, in the individual, of a nucleic acid sequence selected from the group consisting of E1B1 (SEQ ID NO.1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), E28D (SEQ ID NO. 9), and complements thereof, wherein if the presence of the nucleic acid sequence varies from that of a normal individual, the individual has an increased risk of heart disease.

3. The method of claim 2, wherein the nucleic acid sequence is mRNA.

4. The method of claim 2, wherein the detecting step comprising obtaining mRNA from a tissue of the individual and hybridizing the mRNA with nucleic acid a probe that is specific for E1B1, E1B2, E1B3, E1B4, E2B1, E2B2, E28B, E28C, and E28D.

5. The method of claim 4, wherein the tissue is selected from the group consisting of heart muscle, skeletal muscle, and white blood cell.

6. The method of claim 2, wherein the heart disease is arrhythmia or heart failure.

7. The method of claim 2, wherein an increase in E28C and E28D in the individual when compared to the individual without heart disease indicates a risk for heart disease.

8. The method of claim 7, wherein the increase of E28C is about 14 fold.

9. The method of claim 7, wherein the increase of E28D is about 4 fold.

10. The method of claim 2, wherein a decrease in E28B in the individual when compared to the individual without heart disease indicates a risk for heart disease.

11. The method of claim 9, wherein the increase of E28D is about 74.2%.

12. A method of monitoring the treatment of a patient with heart disease comprising the steps of
a. administering a pharmaceutical composition to the patient; and
b. detecting the presence, in the patient, of a nucleic acid sequence selected from the group consisting of E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), E28D (SEQ ID NO. 9), and complements thereof.

13. The method of claim 12, further comprising the step of comparing the result of the assaying step with that of an individual without heart disease, wherein if the two results are similar, the treatment is successful.

14. A method for screening for an agent capable of modulating the onset or progression of heart disease comprising the steps of
a. exposing a cell to the agent; and
b. detecting the presence, in the cell, of a nucleic acid sequence selected from the group consisting of E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), E28D (SEQ ID NO. 9), and complements thereof.

15. The method of claim 19, further comprising the step of comparing the result of the assaying step with that of an individual without heart disease, wherein if the two results are similar, the agent is capable of modulating the onset or progression of heart disease.

16. The method of claim 19, wherein the cell is a cardiac muscle cell, skeletal muscle cell, or a white blood cell.

17. A vector comprising the nucleic acid sequence of E1B1 (SEQ ID NO. 1), E1B2 (SEQ ID NO. 2), E1B3 (SEQ ID NO. 3), E1B4 (SEQ ID NO. 4), E2B1 (SEQ ID NO. 5), E2B2 (SEQ ID NO. 6), E28B (SEQ ID NO. 7), E28C (SEQ ID NO. 8), E28D (SEQ ID NO. 9), or complements thereof.

18. A cell comprising the vector of claim 17.
